SEQUENCE LISTING (24 sequences)

SEQ ID NO: 1 - protein, 451 aa, Artificial Sequence
  Exemplified IgG HC1 (TNFa)
  MISC_FEATURE (451)..(451): Xaa at residue 451 is either K or absent
EVQLVESGGGLVQPGRSLRLSCAASGFTFDDYAMHWVRYAPGKGLEWVSAITWNSGHIDYADSVEGRFTISRDNAKNSLYLQMNSLRAEDTAVYYCAKVSYLSTASSLDYWGRGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPDSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRGDMTKNQVQLTCLVKGFYPSDIAVEWESNGQPENNYKTTPPVLDSDGSFFLASKLTVDKSRWQQGNVFSCSVMHEALHNHYTQKSLSLSPGX

SEQ ID NO: 2 - protein, 214 aa, Artificial Sequence
  Exemplified IgG LC1 (TNFa)
DIQMTQSPSSLSASVGDRVTITCRASQGIRNYLAWYQRKPGDAPKLLIYAASTLQSGVPSRFSGSGSGTDFTLTISSLQPEDVATYYCQRYNRAPYTFGQGTKVEIKRTVAAPSVFIFPPSKEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 445 aa, Artificial Sequence
  Exemplified IgG HC2 (IL23p19)
  MISC_FEATURE (1)..(1): Xaa at residue 1 is either Q or pyroglutamic acid
  MISC_FEATURE (74)..(74): Xaa at residue 74 is either T or E
  MISC_FEATURE (445)..(445): Xaa at residue 445 is either K or absent
XVQLVQSGAEVKKPGSSVKVSCKASGYPFTRYVMHWVRKAPGQGLEWMGYINPYNDGVNYNEEFKGRVTITADXSTSTAYMELSSLRSEDTAVYYCARNWDTGLWGQGTTVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVSTLPPSREEMTKNQVSLMCLVYGFYPSDIAVEWESNGQPENNYKTTPPVLDSDGSFFLYSVLTVDKSRWQQGNVFSCSVMHEALHNHYTQKSLSLSPGX

SEQ ID NO: 4 - protein, 212 aa, Artificial Sequence
  Exemplified IgG LC2 (IL23p19)
RIQMTQSPSSLSASVGDRVTITCKASDHIGKFLTWYQDKPGKAPKLLIYGATSKLTGVPSRFSGSGSGTDFTLTISSLQPEDFATYYCQQYWSTPFTFGGGTKVEIKGQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTEC

SEQ ID NO: 5 - DNA, 1413 bp, Artificial Sequence
  DNA Seq. Encoding Exemplified IgG HC1
atggagacggacactctgctcctgtgggtgctcctgctttgggtaccgggttcaacgggagaggtgcagctggtggagtctgggggaggcttggtacagcctgggaggtccctgagactctcctgtgcagcctctggattcacctttgatgactatgccatgcactgggtccgctacgctccagggaaggggctggagtgggtgtcagctattacttggaatagtggtcacatagactacgcagactccgtggagggccggttcaccatctccagagacaatgccaagaactccctgtatctgcaaatgaacagcctgagagccgaggacacggccgtatattactgtgcgaaagtgagctacctgagtactgcctccagcctggactactggggcagaggaaccctggtgaccgtcagctcagctagcaccaagggcccatcggtcttccccctggcaccctgctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccgactctggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggggggacatgaccaagaaccaggtccagctgacctgcctggtcaaaggcttctatcccagcgacatcgccgtggagtgggagagcaatgggcagccggagaacaactacaagaccacgcctcccgtgctggactccgacggctccttcttcctcgccagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - DNA, 702 bp, Artificial Sequence
  DNA Seq. Encoding Exemplified IgG LC1
atggaaactgacaccctgctgctctgggtactgctcctttgggttcctgggagcacaggcgacatccagatgacccagtctccatcctccctgtctgcatctgtaggagacagagtcaccatcacttgccgggcgagtcagggcattcgcaattatttagcctggtatcagcggaaaccaggggacgctcctaagctcctgatctatgctgcatccactttgcaatcaggggtcccatctcggttcagtggcagtggatctgggacagatttcactctcaccatcagcagcctgcagcctgaagatgttgcaacttattactgtcaacgctataaccgtgccccttacacgttcggccaagggaccaaggtggagatcaagcgtacggtggctgcaccatctgtcttcatcttcccgccatctaaggagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 7 - DNA, 1395 bp, Artificial Sequence
  DNA Seq. Encoding Exemplified IgG HC2
atggaaaccgatacgctcctgctgtgggttctcctcttgtgggtccccggctctaccgggcaggtgcagctggtgcagtctggggctgaggtgaagaagcctgggtcctcggtgaaggtctcctgcaaggcttctggatatcccttcactcgttatgttatgcactgggtgcgaaaggcccctggacaagggcttgagtggatgggatatattaatccttacaatgatggtgtgaactacaatgaggagttcaaaggcagagtcacgattaccgcggacacctccacgagcacagcctacatggagctgagcagcctgagatctgaggacacggccgtgtattactgtgcgagaaactgggacacaggcctctggggccaaggcaccactgtcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtggccaccggcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgagcaccctgcccccatcccgggaggagatgaccaagaaccaggtcagcctgatgtgcctggtctacggcttctatcccagcgacatcgccgtggagtgggagagcaatgggcagccggagaacaactacaagaccacgcctcccgtgctggactccgacggctccttcttcctctacagcgtgctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - DNA, 696 bp, Artificial Sequence
  DNA Seq. Encoding Exemplified IgG LC2
atggagacagacacactcctgctatgggtactgctgctctgggttccaggatccactggtcgcatccagatgacccagtctccatcctccctgtctgcatctgtaggagacagagtcaccatcacttgcaaggcaagtgaccacattggcaaatttttaacttggtatcaggacaaaccagggaaagcccctaagctcctgatctatggtgcaaccagtaagttgactggggtcccatcaaggttcagtggcagtggatctgggacagatttcactctcaccatcagcagtctgcaacctgaagattttgcaacttactactgtcaacagtattggagtactccgttcacgttcggaggggggaccaaggtggaaataaaaggtcagcccaaggctgccccctcggtcactctgttcccgccctcctctgaggagcttcaagccaacaaggccacactggtgtgttacataagtgacttctacccgggagccgtgacagtggcctggaaggcagatagcagccccgtcaaggcgggagtggagaccaccacaccctccaaacaaagcaacaacaagtacgcggcctggagctatctgagcctgacgcctgagcagtggaagtcccacagaagctacagctgccaggtcacgcatgaagggagcaccgtggagaagacagtggcccctacagaatgc

SEQ ID NO: 9 - protein, 121 aa, Artificial Sequence
  Exemplified IgG HCVR1
EVQLVESGGGLVQPGRSLRLSCAASGFTFDDYAMHWVRYAPGKGLEWVSAITWNSGHIDYADSVEGRFTISRDNAKNSLYLQMNSLRAEDTAVYYCAKVSYLSTASSLDYWGRGTLVTVSS

SEQ ID NO: 10 - protein, 107 aa, Artificial Sequence
  Exemplified IgG LCVR1
DIQMTQSPSSLSASVGDRVTITCRASQGIRNYLAWYQRKPGDAPKLLIYAASTLQSGVPSRFSGSGSGTDFTLTISSLQPEDVATYYCQRYNRAPYTFGQGTKVEIK

SEQ ID NO: 11 - protein, 115 aa, Artificial Sequence
  Exemplified IgG HCVR2
  MISC_FEATURE (1)..(1): Xaa at residue 1 is either Q or pyroglutamic acid
  MISC_FEATURE (74)..(74): Xaa at residue 74 is either T or E
XVQLVQSGAEVKKPGSSVKVSCKASGYPFTRYVMHWVRKAPGQGLEWMGYINPYNDGVNYNEEFKGRVTITADXSTSTAYMELSSLRSEDTAVYYCARNWDTGLWGQGTTVTVSS

SEQ ID NO: 12 - protein, 107 aa, Artificial Sequence
  Exemplified IgG LCVR2
RIQMTQSPSSLSASVGDRVTITCKASDHIGKFLTWYQDKPGKAPKLLIYGATSKLTGVPSRFSGSGSGTDFTLTISSLQPEDFATYYCQQYWSTPFTFGGGTKVEIK

SEQ ID NO: 13 - protein, 13 aa, Artificial Sequence
  Exemplified HCDR1
AASGFTFDDYAMH

SEQ ID NO: 14 - protein, 17 aa, Artificial Sequence
  Exemplified HCDR2
AITWNSGHIDYADSVEG

SEQ ID NO: 15 - protein, 14 aa, Artificial Sequence
  Exemplified HCDR3
AKVSYLSTASSLDY

SEQ ID NO: 16 - protein, 11 aa, Artificial Sequence
  Exemplified LCDR1
RASQGIRNYLA

SEQ ID NO: 17 - protein, 8 aa, Artificial Sequence
  Exemplified LCDR2
YAASTLQS

SEQ ID NO: 18 - protein, 9 aa, Artificial Sequence
  Exemplified LCDR3
QRYNRAPYT

SEQ ID NO: 19 - protein, 13 aa, Artificial Sequence
  Exemplified HCDR4
KASGYPFTRYVMH

SEQ ID NO: 20 - protein, 17 aa, Artificial Sequence
  Exemplified HCDR5
YINPYNDGVNYNEEFKG

SEQ ID NO: 21 - protein, 8 aa, Artificial Sequence
  Exemplified HCDR6
ARNWDTGL

SEQ ID NO: 22 - protein, 11 aa, Artificial Sequence
  Exemplified LCDR4
KASDHIGKFLT

SEQ ID NO: 23 - protein, 8 aa, Artificial Sequence
  Exemplified LCDR5
YGATSKLT

SEQ ID NO: 24 - protein, 9 aa, Artificial Sequence
  Exemplified LCDR6
QQYWSTPFT

(19) United States
(12) Patent Application Publication
(10) Pub. No.: US 2019/0040156 A1
(43) Pub. Date: Feb. 7, 2019

(54) ANTI-TNF- / ANTI-IL-23 IgG BISPECIFIC ANTIBODIES

(71) Applicant: Eli Lilly and Company, Indianapolis, IN (US)

(72) Inventors: Stephen J Demarest, San Diego, CA (US); Songqing Na, San Diego, CA (US); Jeffrey S Boyles, Indianapolis, IN (US); Qing Chai, San Diego, CA (US)

(21) Appl. No.: 16/046,282

(22) Filed: Jul. 26, 2018

Related U.S. Application Data
(60) Provisional application No. 62/540,182, filed on Aug. 2, 2017.

(51) Int. Cl. C07K 16/46; C07K 16/24; A61P 37/06; A61P 19/02

(52) U.S. Cl. CPC C07K 16/468; C07K 16/241; C07K 16/244; A61P 37/06; A61P 19/02; A61K 2039/505; C07K 2317/55; C07K 2317/52; C07K 2317/732; C07K 2317/734; C07K 2317/92; C07K 2317/31

(57) ABSTRACT

IgG bispecific antibodies are provided that bind tumor necrosis factor alpha (TNFα) and the p19 subunit of interleukin-23 (IL-23p19) and are characterized as having high affinity and simultaneous neutralizing properties to both TNFα and IL-23. The bispecific antibodies of the invention are useful for treating various autoimmune diseases including inflammatory bowel disease, such as Crohn's disease and ulcerative colitis, psoriasis, psoriatic arthritis, and Hidradenitis suppurativa.

The present invention is in the field of medicine, particularly in the novel field of IgG bispecific antibodies capable of perturbing two distinct therapeutic targets. The IgG bispecific antibodies of the present invention are directed against Tumor Necrosis Factor alpha (TNFα) and the p19 subunit of Interleukin-23 (IL23p19) and are expected to be useful in treating autoimmune diseases including inflammatory bowel disease (IBD), such as Crohn's disease (CD) and ulcerative colitis (UC), psoriasis (Pₛ0), psoriatic arthritis (PsA), and Hidradenitis suppurativa (HS).

Autoimmune diseases arise from the body's production of an immune response against its own tissue. Autoimmune diseases are often chronic and can be debilitating and even life-threatening. IBD, which generically represents a group of disorders such as CD and UC, is a common chronic relapsing autoimmune disease characterized pathologically by intestinal inflammation and epithelial injury. Other forms of chronic autoimmune diseases, such as Ps0, PsA and HS, may affect the axial and/or peripheral skeleton.

Interleukin 23 (IL-23) is a heterodimeric cytokine believed to be important in the activation of a range of inflammatory cells involved in the induction of chronic inflammation. IL-23, which is an upstream regulator of IL-6, IL-17, GM-CSF and IL-22, is composed of a p19 subunit (IL23p19) covalently paired to a p40 subunit (the p40 subunit is shared with cytokine IL-12). Additionally, IL-23 has been implicated as playing an important role in both memory/pathogenic T-cell inflammatory response as well as playing a role in the regulation of innate lymphoid cell inflammatory activity. There is evidence that IL-23 regulation of the cytokines IL-6, IL-17, GM-CSF and IL-22 is associated with inflammatory diseases including IBD and other autoimmune diseases.

Tumor Necrosis Factor alpha (TNFα) is a pleiotropic homotrimeric cytokine which is primarily secreted by monocytes and macrophages, but also known to be produced by CD4⁺ and CD8⁺ peripheral blood T lymphocytes. TNFα is expressed in both a soluble and transmembrane form (the membrane-bound precursor form can be proteolytically cleaved into a soluble homotrimer by metalloproteinase TNF alpha converting enzyme (TACE)). TNFα is believed to play a role in the regulation of immune cells and be important in systemic inflammation, specifically in acute phase inflammatory reactions. Excess amounts of TNFα have been associated with various forms of autoimmune diseases, including Ps0 and CD.

Current FDA approved treatments for autoimmune diseases such as IBD, including UC and CD, include corticosteroids, often used to treat acute inflammation, as well as bioproducts (such as REMICADE® and HUMIRA®). However, current biologic treatments for IBD, such as UC and CD, offer a remission of symptoms of only 10-30 percent above placebo rates and endoscopic and clinical remissions are often not achieved at the same rate in the same patients. In addition to a large percentage of patients being nonresponsive to currently available treatments, loss of response to TNFα neutralization occurs in between 23 and 46 percent of patients following 12 months of treatment.

Likewise, only a minority of PsA and Ps0 patients are treated with an approved biologic (which includes REMICADE® and HUMIRA®, as well as STELARA® which targets the shared p40 subunit of cytokines IL-12 and IL-23). Current treatments have demonstrated efficacy for reducing symptoms and slowing progression of some forms of Pₛ0 in a subset of patients. Similarly, current biologic treatments for PsA have demonstrated efficacy in subsets of patients with 25 to 45% of articular PsA patients achieving an American College of Rheumatology score of 50 by 24 weeks of treatment. However, no single biologic has demonstrated overwhelming efficacy in a significant amount of patients and lack of, or loss of, response to current biologics remains an issue.

In addition to the considerable negative impact on quality of life imposed by autoimmune diseases such as IBD (including CD and UC), Ps0, HS and PsA), current treatment regimens can be quite burdensome. Thus, there remains a need for alternative therapies for treatment of autoimmune diseases, including IBD (such as CD and UC), Ps0, HS and PsA. Preferably, such alternative therapies will address one or more of the concerns not addressed by current treatment options such as demonstrating efficacy in a larger percentage of patients non-responsive to currently available treatments, providing a sufficient response in a larger percent of patients (currently not acceptably treated), reducing the loss of response in patients and/or providing a less burdensome treatment regimen.

One approach to such alternative therapies may include the targeting of multiple targets. Targeting multiple biological targets may be accomplished by co-administration or combination use of two different bioproducts (e.g., two antibodies capable of perturbing different therapeutic targets). However, co-administration or combination use of separate bioproducts presents both practical and commercial challenges. For example, while two injections of separate bioproducts may permit flexibility of dose amounts and timing, it is inconvenient to patients and providers for compliance and pain. Additionally, while a co-formulation might provide some flexibility of dose amounts, it is often quite challenging or impossible to find formulation conditions having acceptable viscosity in solution (at relatively high concentration) and that permit chemical and physical stability of both antibodies due to different molecular characteristics of the two antibodies. Additionally, co-administration and co-formulation involve the additive costs of two different drug therapies which can increase patient and/or payor costs.

Bispecific antibodies, single agents capable of perturbing two distinct targets, have been proposed as a means for addressing the limitations attendant with co-administration or co-formulation of separate antibody agents. Bispecific antibodies integrate the binding activities of two separate antibody therapeutics into a signal agent, thus providing potential cost and convenience benefits to the patient. In some circumstances, bispecific antibodies may also elicit synergistic or novel activities beyond what co-administration or co-formulation combinations can achieve. However, due to complexity of manufacturing and/or immunogenicity, bispecific antibodies have in most cases not been ideal.

Multiple formats for bispecific antibodies have been proposed. For example, U.S. Patent Publication Number 2012/0251541 A1 discloses bispecific antibodies targeting TNFα and IL23/IL12 which possess a cross-over dual variable configuration. Similarly, U.S. Pat. No. 7,612,181 discloses bispecific antibodies targeting TNFα and IL23 which have a dual variable domain configuration. Other bispecific antibody formats involve the conjugation of two antibodies, or fragments thereof, via chemical conjugation (see Brennan, M., et al., Science, 1985. 229(4708): p. 81-3) or via a bifunctional crosslinker (see Glennie, M. J., et al., J Immunol, 1987. 139(7): p. 2367-75). Even further bispecific antibody formats include single chain Fv (scFv) fragments composed of heavy and light chain variable regions tethered by flexible or structured linkers. One or more scFv fragments which bind a particular target are linked to another moiety, for example a separate scFv or an IgG antibody, which binds a separate target (for example, U.S. Pat. No. 9,718,884 B2 which discloses an anti-TNF/anti-IL23p19 IgG-scFv bispecific antibody). Each of the above bispecific antibody formats, however, has limitations in that they lack the archetypical Fab architecture which provides stabilizing interactions of the heavy chain and light chain constant domains (i.e., C_{H}1 and C_{L}, respectively) which can improve thermal stability, solubility, reduce the potential for insoluble aggregation during manufacturing, and/or improve drug delivery characteristics such as viscosity. Furthermore, each of the above bispecific antibody formats deviates from the archetypical IgG structure which may also lead to increased immunogenicity risk, by way of presentation of neo-epitopes not observed with archetypical IgG structure or varied immune complex formation, and altered effector function.

Therefore, a need still exists for a single bispecific antibody that neutralizes both human TNFα and human IL-23, where the bispecific antibody specifically targets the p19 subunit of human IL-23 and does not specifically neutralize IL-12 (which shares a common p40 subunit with IL-23). It is desirable that such bispecific antibody possess the archetypical IgG (including archetypical Fab) architecture and be thermally and physically stable, exhibit low insoluble aggregation, reduced viscosity during drug delivery, neutralize human TNFα and human IL23p19, and not present an increased immunogenicity risk associated with neo-epitopes associated with bispecific antibodies deviating from the archetypical IgG structure. Also, it is desirable that effector function of such bispecific antibodies, when comprising IgG1 or IgG3 heavy chains, be retained or enhanced. Further, it is desirable to provide a pharmaceutical composition including a single bispecific antibody that neutralizes both human TNFα and human IL23p19, thereby avoiding the challenges of finding formulation conditions that must satisfy the different molecular characteristics of two different, separate antibodies. The present invention therefore provides bispecific antibodies retaining the immunoglobulin G (“IgG”) antibody architecture (“IgG bispecific antibodies”) directed against TNFα and IL23p19, seeking to address one or more of the above mentioned problems and provide a sufficient response in those patients who fail to have a sufficient response and/or are non-responsive in currently available TNFα and IL23 treatments alone.

The present invention addresses one or more of the above problems by providing an IgG bispecific antibody comprising a first heavy chain (HC1) first light chain (LC1) pair (HC1-LC1), a second heavy chain (HC2) second light chain (LC2) pair (HC2-LC2), and a HC1-HC2 interface, wherein HC1 forms at least one inter-chain disulfide bond with LC1, HC2 forms at least one inter-chain disulfide bond with LC2, and HC1 forms at least two inter-chain disulfide bond with HC2, and further wherein HC1-LC1 binds to TNFα and HC2-LC2 binds to IL23p19. The present invention also provides a HC1-HC2 interface, as well as a HC1-LC1 pair and a HC2-LC2 pair which improve assembly of IgG bispecific antibodies.

In accordance with the present invention, a HC1-HC2 interface is provided which achieves improved heterodimerization of distinct heavy chains (HC1 and HC2) by introducing specific mutations in the Fc domain of the respective heavy chains. In particular embodiments of the present invention, IgG bispecific antibodies of the present invention comprise a HC1 having a first human heavy chain IgG1 Fc domain comprising: glycine substituted at residue 356 (corresponding to glycine at residue 360 of SEQ ID NO: 1); aspartic acid substituted at residue 357 (corresponding to aspartic acid at residue 361 of SEQ ID NO: 1); glutamine substituted at residue 364 (corresponding to glutamine at residue 368 of SEQ ID NO: 1); and alanine substituted at residue 407 (corresponding to alanine at residue 411 of SEQ ID NO:1). Embodiments of the IgG bispecific antibodies of the present invention comprise a HC2 having a second human heavy chain IgG1 Fc domain comprising: serine substituted at residue 349 (corresponding to serine at residue 347 of SEQ ID NO:3); methionine substituted at residue 366 (corresponding to methionine at residue 364 of SEQ ID NO:3); tyrosine substituted at residue 370 (corresponding to tyrosine at residue 368 of SEQ ID NO:3); and valine substituted at residue 409 (corresponding to valine at residue 407 of SEQ ID NO:3). Residue numbering of the Fc domain of HC1 and HC2 is based on the EU Numbering convention; residue numbering in relation to SEQ ID NOs: 1 and 3 is linear.

Additionally, distinct HC1-LC1 and HC2-LC2 pairs (or arms) are provided which achieve improved assembly and heterodimerization of the IgG bispecific antibody, but maintaining affinity, by introducing specific mutations in the Fab domain of the heavy and light chains, respectively. In particular embodiments, the IgG bispecific antibodies of the present invention comprise a HC1-LC1 pair comprising a Fab domain, wherein HC1 comprises: tyrosine substituted at residue 39 (corresponding to tyrosine at residue 39 of SEQ ID NO:1); arginine substituted at residue 105 (corresponding to arginine at residue 113 of SEQ ID NO: 1); cysteine substituted at residue 127 (corresponding to cysteine at residue 135 of SEQ ID NO: 1); aspartic acid substituted at residue 228 (corresponding to aspartic acid at residue 222 of SEQ ID NO: 1); and glycine substituted at residue 230 (corresponding to glycine at residue 224 of SEQ ID NO: 1), and wherein the LC1 comprises: arginine substituted at residue 38 (corresponding to arginine at residue 38 of SEQ ID NO:2); aspartic acid substituted at residue 42 (corresponding to aspartic acid at residue 42 of SEQ ID NO:2); and lysine substituted at residue 122 (corresponding to lysine at residue 122 of SEQ ID NO:2). Furthermore, embodiments of the IgG bispecific antibodies of the present invention comprise a HC2-LC2 pair comprising a Fab domain, wherein HC2 comprises: lysine substituted at residue 39 (corresponding to lysine at residue 39 of SEQ ID NO:3); glutamic acid substituted at residue 62 (corresponding to glutamic acid at residue 62 of SEQ ID NO:3); alanine substituted at residue 172 (corresponding to alanine at residue 166 of SEQ ID NO:3); and glycine substituted at residue 174 (corresponding to glycine at residue 168 of SEQ ID NO:3), and wherein the LC2 comprises arginine substituted at residue 1 (corresponding to arginine at residue 1 of SEQ ID NO:4); aspartic acid substituted at residue 38 (corresponding to aspartic acid at residue 38 of SEQ ID NO:4); tyrosine substituted at residue 135 (corresponding to tyrosine at residue 136 of SEQ ID NO:4); and tryptophan substituted at residue 176 (corresponding to tryptophan at residue 176 of SEQ ID NO:4). In even more particular embodiments of the IgG bispecific antibodies of the present invention HC2 comprises threonine substituted at residue 74 of SEQ ID NO:3 (corresponding to threonine at residue 74 of SEQ ID NO:3). Residue numbering of the Fab domain of HC1, LC1, HC2 and LC2 is based on Kabat residue numbering convention; residue numbering in relation to SEQ ID NOs:1, 2, 3 and 4 is linear.

In particular embodiments, the present invention provides IgG bispecific antibodies comprising:
a) a first heavy chain (HC1) comprising a heavy chain variable region (HCVR1), wherein HCVR1 comprises heavy chain complementarity determining regions (HCDR) 1-3 wherein the amino acid sequence of HCDR1 is SEQ ID NO:13, the amino acid sequence of HCDR2 is SEQ ID NO: 14, and the amino acid sequence of HCDR3 is SEQ ID NO:15;
b) a first light chain (LC1) comprising a light chain variable region (LCVR1), wherein LCVR1 comprises light chain complementarity determining regions (LCDR) 1-3 wherein the amino acid sequence of LCDR1 is SEQ ID NO:16, the amino acid sequence of LCDR2 is SEQ ID NO:17, and the amino acid sequence of LCDR3 is SEQ ID NO:18;
c) a second heavy chain (HC2) comprising a heavy chain variable region (HCVR2), wherein HCVR2 comprises heavy chain complementarity determining regions (HCDR) 4-6 wherein the amino acid sequence of HCDR4 is SEQ ID NO:19, the amino acid sequence of HCDR5 is SEQ ID NO:20, and the amino acid sequence of HCDR6 is SEQ ID NO:21; and
d) a second light chain (LC2) comprising a light chain variable region (LCVR2), wherein the LCVR2 comprises light chain complementarity determining regions (LCDR) 4-6 wherein the amino acid sequence of LCDR4 is SEQ ID NO:22, the amino acid sequence of LCDR5 is SEQ ID NO:23, and the amino acid sequence of LCDR6 is SEQ ID NO:24,
   wherein HC1 forms at least one inter-chain disulfide bond with LC1 (HC1-LC1 pair), HC2 forms at least one inter-chain disulfide bond with LC2 (HC2-LC2 pair), and HC1 forms at least two inter-chain disulfide bonds with HC2, and wherein the HC1-LC1 pair binds to human TNFα and the HC2-LC2 pair binds to human IL23p19. Particular embodiments include HC2 comprising threonine at residue 74 (residue numbering based on the Kabat convention).

According to some particular embodiments of the IgG bispecific antibodies of the present invention,
a) HC1 comprises tyrosine at residue 39, arginine at residue 105, cysteine at residue 127, aspartic acid at residue 228, and glycine at residue 230 (residue numbering based on Kabat),
b) LC1 comprises arginine at residue 38, aspartic acid at residue 42, and lysine at residue 122 (residue numbering based on Kabat),
c) HC2 comprises lysine at residue 39, glutamic acid at residue 62, alanine at residue 172, and glycine at residue 174 (residue numbering based on Kabat), and
d) LC2 comprises arginine at residue 1, aspartic acid at residue 38, tyrosine at residue 135, and tryptophan at residue 176 (residue numbering based on Kabat).
   According to some such embodiments, the amino acid sequence of HCVR1 is SEQ ID NO:9, the amino acid sequence of LCVR1 is SEQ ID NO:10, the amino acid sequence of HCVR2 is SEQ ID NO: 11, and the amino acid sequence of LCVR2 is SEQ ID NO:12. In more particular embodiments, HCVR2 comprises threonine at residue 74 of SEQ ID NO:11.

Further, according to some embodiments of the IgG bispecific antibodies of the present invention, HC1 and HC2 are human IgG1 heavy chains, LC1 is a human kappa light chain and LC2 is a human lambda light chain variable region and a human light chain constant region. According to particular embodiments,
a) HC1 comprises a human IgG Fc domain comprising glycine at residue 356, aspartic acid at residue 357, glutamine at residue 364, and alanine at residue 407 (residue numbering based on the EU Numbering convention), and
b) HC2 comprises a human IgG Fc domain comprising serine at residue 349, methionine at residue 366, tyrosine at residue 370, and valine at residue 409 (residue numbering based on the EU Numbering convention).
   In even more particular embodiments, the amino acid sequence of HC1 is SEQ ID NO:1, the amino acid sequence of LC1 is SEQ ID NO:2, the amino acid sequence of HC2 is SEQ ID NO:3, and the amino acid sequence of LC2 is SEQ ID NO:4. In even more particular embodiments, HC2 comprises threonine at residue 74 of SEQ ID NO:3.

The present invention also provides a method of treating autoimmune diseases comprising administering to a patient in need thereof an effective amount of an IgG bispecific antibody of the present invention. According to some embodiments, the present invention provides a method of treating IBD, such as CD and UC, comprising administering to a patient in need thereof a therapeutically effective amount of an IgG bispecific antibody of the present invention. According to further embodiments, the present invention provides a method of treating one or more of Ps0, PsA and HS comprising administering to a patient in need thereof a therapeutically effective amount of an IgG bispecific antibody of the present invention.

The present invention also provides an IgG bispecific antibody of the present invention for use in therapy. According to some embodiments, the present invention provides an IgG bispecific antibody of the present invention for use in the treatment of an autoimmune disease including IBD, such as CD and UC. Further embodiments of the present invention provide an IgG bispecific antibody of the present invention for use in the treatment of an autoimmune disease including one or more of Ps0, PsA and HS.

The present invention also provides a pharmaceutical composition comprising an IgG bispecific antibody of the present invention and one or more pharmaceutically acceptable carriers, diluents or excipients.

Embodiments of the present invention also comprise use of an IgG bispecific antibody of the present invention in the manufacture of a medicament for the treatment of one or more of UC and CD. Additional embodiments of the present invention comprise use of an IgG bispecific antibody of the present invention in the manufacture of a medicament for the treatment of one or more of Ps0, PsA and HS.

The present invention also provides a DNA molecule comprising a polynucleotide sequence encoding a polypeptide chain comprising a HC1 of the IgG bispecific antibody of present invention. According to more particular embodiments, the amino acid sequence of the encoded HC1 is SEQ ID NO:1. According to some such embodiments, the nucleotide sequence of the DNA molecule is SEQ ID NO:5. Embodiments of the present invention also provide a DNA molecule comprising a polynucleotide sequence encoding a polypeptide chain comprising a LC1 of the IgG bispecific antibody of the present invention. According to more particular embodiments, the amino acid sequence of the encoded LC1 is SEQ ID NO:2. According to some such embodiments, the nucleotide sequence of the DNA molecule is SEQ ID NO:6. Further, embodiments of the present invention also provide a DNA molecule comprising a polynucleotide sequence encoding a polypeptide chain comprising a HC2 of the IgG bispecific antibody of present invention. According to more particular embodiments, the amino acid sequence of the encoded HC2 is SEQ ID NO:3. In some particular embodiments, HC2 comprises tyrosine at residue 74 of SEQ ID NO:3. According to some such embodiments, the nucleotide sequence of the DNA molecule is SEQ ID NO:7. Even further embodiments of the present invention also provide a DNA molecule comprising a polynucleotide sequence encoding a polypeptide chain comprising a LC2 of the IgG bispecific antibody of present invention. According to more particular embodiments, the amino acid sequence of the encoded LC2 is SEQ ID NO:4. According to some such embodiments, the nucleotide sequence of the DNA molecule is SEQ ID NO:8.

According to some embodiments of the DNA molecules of the present invention, the polynucleotide sequence encoding the HC1 of the IgG bispecific antibody of present invention also comprises a polynucleotide sequence encoding the LC1 of the IgG bispecific antibody of the present invention. According to some embodiments, the DNA molecule comprising the polynucleotide sequence encoding the HC2 of the IgG bispecific antibody of present invention also comprises a polynucleotide sequence encoding the LC2 of the IgG bispecific antibody of the present invention. According to even further embodiments, the DNA molecule comprising the polynucleotide sequence encoding the HC1 and the polynucleotide sequence encoding the LC1 of the IgG bispecific antibody of the present invention further comprises the polynucleotide sequence encoding the HC2 and the polynucleotide sequence encoding the LC2 of the IgG bispecific antibody of the present invention.

Embodiments of the present invention also provide a mammalian cell comprising a DNA molecule of the present invention, wherein the cell is capable of expressing an IgG bispecific antibody of the present invention, said IgG bispecific antibody comprising a HC1-LC1 pair and a HC2-LC2 pair, wherein the HC1-LC1 pair binds to human TNFα and the HC2-LC2 pair binds to human IL23p19.

The present invention also provides a process for producing an IgG bispecific antibody of the present invention, the process comprising cultivating a mammalian cell of the present invention under conditions such that the IgG bispecific antibody is expressed, and recovering the expressed IgG bispecific antibody. The present invention also provides an IgG bispecific antibody according to the present invention produced by said process.

### Definitions

When used herein the term “IgG bispecific antibody” refers to a heterodimeric IgG molecule, or fragment thereof, which retains the immunoglobulin G (“IgG”) antibody architecture and comprises a discrete first heavy chain (HC1), a discrete first light chain (LC1), a discrete second heavy chain (HC2), and a discrete second light chain (LC2), wherein HC1 forms at least one inter-chain disulfide bond with LC1 (forming a HC1-LC1 pair, also referred to herein as an “arm” or “HC1-LC1 arm”), HC2 forms at least one inter-chain disulfide bond with LC2 (forming a HC2-LC2 pair, also referred to herein as an “arm” or “HC2-LC2 arm”), and HC1 forms at least two inter-chain disulfide bonds with HC2. A representation of an IgG bispecific antibody of the present invention is provided in the following schematic:

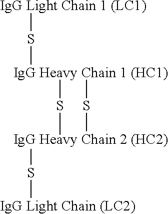

The HC1-LC1 pair of the IgG bispecific antibodies of the present invention exhibit selective monovalent binding to TNFα, whereas the HC2-LC2 pair of the IgG bispecific antibodies of the present invention exhibit selective monovalent binding to IL23p19.

When used herein, the term “immunoglobulin G antibody” (IgG), refers to an immunoglobulin molecule comprised of four discrete polypeptide chains; two heavy chains (HC1 and HC2) and two light chains (LC1 and LC2) interconnected by disulfide bonds as described herein. The amino-terminal portion of each of the four polypeptide chains includes a variable region of about 100-120 or more amino acids primarily responsible for antigen recognition. Each of the four polypeptide chains also comprise constant regions.

Light chains (LC) of the IgG bispecific antibodies of the present invention are classified as kappa or lambda and characterized by a particular constant region as known in the art. According to particular embodiments of the IgG bispecific antibodies of the present invention, LC2 is comprised of a human kappa variable domain and a human lambda constant region whereas LC1 is comprised of a human kappa variable domain and constant region. As referred to herein, human kappa and human lambda refer to the origin of the sequences comprising the respective LC variable domains or constant regions and further include the specifically engineered amino acid modifications disclosed here. The heavy chains (HC) of the IgG bispecific antibodies according to the present invention are classified as gamma, which defines the isotype (e.g., an IgG). The isotype may be further divided into subclasses (e.g., IgG1, IgG2, IgG3, and IgG4). Each HC is comprised of an N-terminal heavy chain variable region (“HCVR”) and a heavy chain constant region (CH) responsible for effector function. The CH for IgG, according to the present invention, is comprised of three domains (CH1, CH2, and CH3). Each light chain of IgG bispecific antibodies of the present invention is comprised of a light chain variable region (LCVR) and a light chain constant region (CL). The HCVR and LCVR regions can be further subdivided into regions of hypervariability, termed complementarity determining regions (CDRs), interspersed with regions that are more conserved, termed framework regions (FR). Each HCVR and LCVR of the IgG bispecific antibodies of the present invention are composed of three CDRs and four FRs, arranged from amino-terminus to carboxy-terminus in the following order: FR1-1, CDR1, FR1-2, CDR2, FR1-3, CDR3, FR1-4. As described herein the 3 CDRs of the HC1 are referred to as “HCDR1, HCDR2 and HCDR3”; the 3 CDRs of HC2 are referred to as “HCDR4, HCDR5 and HCDR6”; the 3 CDRs of LC1 are referred to as “LCDR1, LCDR2 and LCDR3”; and the 3 CDRs of LC2 are referred to as “LCDR4, LCDR5 and LCDR6.” The CDRs contain most of the residues which form specific interactions with the antigen. The functional ability of an HC-LC pair of the IgG bispecific antibodies to bind a particular antigen is largely influenced by the six CDRs comprising each HC-LC pair.

The variable regions of each HC-LC pair of the IgG bispecific antibodies according to the present invention form an antigen-binding site. According to the present invention, the IgG bispecific antibodies have two antigen binding sites, one comprised of the HC1-LC pair and one comprised of the HC2-LC2 pair. As used herein, the “antigen-binding portion” or “antigen-binding site” or “antigen-binding region” or “antigen-binding fragment” refers interchangeably to that portion of an IgG bispecific antibody, within a variable region of a HC1-LC1 or HC2-LC2 pair, respectively, which contains the amino acid residues that interact with an antigen and confer to the IgG bispecific antibody specificity and affinity for a respective antigen. This IgG bispecific antibody variable region also includes the framework amino acid residues necessary to maintain the proper conformation of the antigen-binding residues. Preferably, the framework regions of the IgG bispecific antibodies of the invention are of human origin or substantially of human origin.

A “parent antibody” or “parental antibody,” as used interchangeably herein, is an antibody encoded by an amino acid sequence which is used in the preparation of one HC-LC pair of IgG bispecific antibodies of the present invention, for example through amino acid substitutions and structural alteration. A parental antibody may be a murine, chimeric, humanized or human antibody.

The terms “Kabat”, “Kabat numbering” or “Kabat labeling” are used interchangeably herein. These terms, which are recognized in the art, refer to a system of numbering amino acid residues which are more variable (i.e., hypervariable) than other amino acid residues in the heavy and light chain variable regions of an antibody (Kabat, et al., Ann. N.Y. Acad Sci. 190:382-93 (1971); Kabat et al., Sequences of Proteins of Immunological Interest, Fifth Edition, U.S. Department of Health and Human Services, NIH Publication No. 91-3242 (1991)).

The terms “North numbering” or “North labeling” are used interchangeably herein. These terms, which are recognized in the art, refer to a system of numbering amino acid residues which are more variable (i.e., hypervariable) than other amino acid residues in the heavy and light chain variable regions of an antibody and is based, at least in part, on affinity propagation clustering with a large number of crystal structures, as described in (North et al., A New Clustering of Antibody CDR Loop Conformations, Journal of Molecular Biology, 406:228-256 (2011).

The terms “EU numbering” or “EU labeling” are used interchangeably herein. These terms, which are recognized in the art, refer to a system of numbering amino acid residues to heavy chain constant domains based on the International Immunogenetics Information System® available at www.imgt.org. EU numbering is described at Kabat, et al., Sequences of Proteins of Immunological Interest, Vol. 5, Fifth Edition. pp. 2719 (1992).

The terms “patient,” “subject,” and “individual,” used interchangeably herein, refer to an animal, preferably humans. In certain embodiments, the subject, preferably a human, is further characterized with a disease or disorder or condition (e.g., an autoimmune disorder) that would benefit from a decreased level or decreased bioactivity of one, or preferably both, of IL-23 and TNFα. In another embodiment the subject, preferably a human, is further characterized as being at risk of developing a disorder, disease or condition that would benefit from a decreased level or decreased bioactivity of both IL-23 and TNFα.

### IgG Bispecific Antibody Engineering

IgG bispecific antibodies of the present invention are heterodimeric in that each HC-LC pair, or arm, of the antibody exhibits selective monovalent binding to its cognate antigen due to two different HCs and two different LCs forming the antibody: one arm (HC-LC pair) of the antibody binds human TNFα, and the other arm binds the p19 subunit of human IL-23. However, the ability to generate bispecific antibodies with IgG architecture has been a long-standing challenge in antibody engineering. One proposal for generating IgG bispecific antibodies entails co-expression of nucleic acids encoding two distinct HC-LC pairs which, when expressed, assemble to form a single antibody comprising two distinct Fabs. Challenges with this approach remain however.

Specifically, the expressed polypeptides of each desired Fab must assemble with good specificity to reduce generation of mis-matched byproducts, and the resulting heterotetramer must assemble with good stability. Procedures for directing assembly of particular HC-HC pairs by introducing modifications into regions of the HC-HC interface have been disclosed in the art. (See Klein et al., mAbs, Vol. 4, No. 6, pages 1-11, 2012; Carter et al., J. Immunol. Methods, Vol. 248, pages 7-15, 2001; Gunasekaran, et al., J. Biol. Chem., Vol. 285, pages 19637-19646, 2010; Zhu et al., Protein Science, Vol. 6, pages 781-788, 1997; and Igawa et al., Protein Eng. Des. Sel., Vol. 23 pages 667-677, 2010). However, challenges remain in engineering heterodimeric bispecific antibodies possessing a distinct HC-HC interface and the IgG bispecific antibodies of the present invention have been engineered to overcome these challenges and drive proper assembly thereof.

Initial attempts in constructing an IgG bispecific antibody according to the present invention may include a parental anti-TNFα antibody (for example, adalimumab, as described in U.S. Pat. No. 6,090,382) and a parental anti-IL-23 antibody (for example, as described in U.S. Pat. No. 7,872,102), or may include a parental bispecific antibody targeting TNFα and IL-23 (for example, as described in U.S. Patent Publication Number 2016/0122429 A1). However, initial attempts for constructing IgG bispecific antibodies according to the present invention, even in view of parental antibodies as described above, suffer from one or more of the chemical and physical problems described herein. Extensive engineering, including chemical and physical protein engineering, was conducted to arrive at the IgG bispecific antibodies of the present invention.

For example, parental anti-TNFα and anti-IL-23 antibodies, as described above, have human kappa light chain constant regions. Modifications to the anti-IL-23 arm of the IgG bispecific antibodies of the present invention were identified, whereby the light chain constant region was altered to a human lambda light chain constant region resulting in improvement of assembly of the IgG bispecific antibodies of the present invention. However, modification of the light chain constant region of the anti-TNFα arm (from human kappa light chain, as in parental anti-TNFα antibodies, to human lambda light chain) of IgG bispecific antibodies provided herein did not result in improved stability. Additionally, a number of modifications, engineered in both the Fab and Fc domains of both the anti-TNFα arm and the anti-IL-23 arm of IgG bispecific antibodies provided herein were identified which improve chemical and physical stability and drive heterodimeric assembly. For example, an engineered modification whereby the Fab domain of HC2 possesses threonine at residue 74 (residue numbering based on Kabat) enhances stability and improves drug delivery characteristics, such as viscosity, over constructs incorporating parental anti-IL-23 antibodies into the IgG bispecific antibodies.

Further, embodiments of IgG bispecific antibodies of the present invention contain an Fc portion which is derived from human IgG₁. IgG₁ is well known to bind to the proteins of the Fc-gamma receptor family (FcγR) as well as C1q. Interaction with these receptors can induce complement-dependent cytotoxicity (CDC). In some embodiments, the IgG bispecific antibodies of the present invention possess enhanced CDC activity as compared to parental TNFα antibodies (adalimumab, as described in U.S. Pat. No. 6,090,382).

### IgG Bispecific Antibody Binding

The IgG bispecific antibodies of the present invention bind both human TNFα and human IL23p19 and neutralize at least one human TNFα bioactivity and at least one human IL-23 bioactivity in vitro and/or in vivo. The IgG bispecific antibodies of the present invention are potent inhibitors of IL-23 in the presence and absence of TNFα in vitro. The IgG bispecific antibodies of the present invention are potent inhibitors of both soluble and membrane-bound TNFα in the presence and absence of IL-23 in vitro.

The IgG bispecific antibodies of the invention are further characterized as having a binding affinity (K_{D}) for human TNFα in the range of 49±9.0 pM and human IL23p19 in the range of 245±8 pM at 37° C. The IgG bispecific antibodies effectively neutralize soluble as well as membrane-bound TNFα and this neutralization is not affected by the presence of saturating amounts of human IL-23. The IgG bispecific antibodies effectively neutralize human IL-23 and this neutralization is not affected by the presence of saturating amounts of human TNFα.

### IgG Bispecific Antibody Expression

Expression vectors capable of directing expression of genes to which they are operably linked are well known in the art. Expression vectors can encode a signal peptide that facilitates secretion of the polypeptide(s) from a host cell. The signal peptide can be an immunoglobulin signal peptide or a heterologous signal peptide. Each of the expressed polypeptides may be expressed independently from different promoters to which they are operably linked in one vector or, alternatively, may be expressed independently from different promoters to which they are operably linked in multiple vectors. The expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences. Exemplary suitable vectors for use in preparing fusion compounds of the present invention include vectors available from Lonza Biologics such as pEE 6.4 and pEE 12.4 (for expressing the second polynucleotide sequence for example).

A particular DNA polynucleotide sequence encoding an exemplified HC1 of an IgG bispecific antibody of the present invention (having the amino acid sequence SEQ ID NO:1) is provided by SEQ ID NO:5 (the DNA polynucleotide sequence provided by SEQ ID NO:5 also encodes a N-terminal signal peptide and a C-terminal lysine cleaved post-translationally). A particular DNA polynucleotide sequence encoding an exemplified LC1 of an IgG bispecific antibody of the present invention (having the amino acid sequence SEQ ID NO:2) is provided by SEQ ID NO:6 (the DNA polynucleotide sequence provided by SEQ ID NO:6 also encodes a N-terminal signal peptide). A particular DNA polynucleotide sequence encoding an exemplified HC2 of an IgG bispecific antibody of the present invention (having the amino acid sequence SEQ ID NO:3) is provided by SEQ ID NO:7 (the DNA polynucleotide sequence provided by SEQ ID NO:7 also encodes a N-terminal signal peptide and a C-terminal lysine cleaved post-translationally). A particular DNA polynucleotide sequence encoding an exemplified LC2 of an IgG bispecific antibody of the present invention (having the amino acid sequence SEQ ID NO:4) is provided by SEQ ID NO:8 (the DNA polynucleotide sequence provided by SEQ ID NO:8 also encodes a N-terminal signal peptide).

A host cell refers to cells stably or transiently transfected, transformed, transduced or infected with one or more expression vectors expressing a HC1, HC2, LC1 and a LC2 polypeptide chain of the IgG bispecific antibodies of the present invention. Creation and isolation of host cell lines producing IgG bispecific antibodies of the present invention can be accomplished using standard techniques known in the art. Mammalian cells are preferred host cells for expression of IgG bispecific antibodies of the present invention. Particular mammalian cells are HEK 293, NS0, DG-44, and CHO. Preferably, the IgG bispecific antibodies are secreted into the medium in which the host cells are cultured, from which the IgG bispecific antibodies can be recovered or purified by conventional techniques. For example, the medium may be applied to and eluted from a Protein A or G column using conventional methods. Soluble aggregate and multimers may be effectively removed by common techniques, including size exclusion, hydrophobic interaction, ion exchange, or hydroxyapatite chromatography. The product may be immediately frozen, for example at −70° C., refrigerated, or may be lyophilized. Various methods of protein purification may be employed and such methods are known in the art and described, for example, in Deutscher, Methods in Enzymology 182: 83-89 (1990) and Scopes, Protein Purification: Principles and Practice, 3rd Edition, Springer, N.Y. (1994).

### Therapeutic Uses

As used herein, “treatment” and/or “treating” are intended to refer to all processes wherein there may be a slowing, interrupting, arresting, controlling, or stopping of the progression of the disorders described herein, but does not necessarily indicate a total elimination of all disorder symptoms. Treatment includes administration of an IgG bispecific antibody of the present invention for treatment of a disease or condition in a mammal, particularly in a human, that would benefit from a decreased level of TNFα and/or IL-23 or decreased bioactivity of TNFα and/or IL-23, and includes: (a) inhibiting further progression of the disease, i.e., arresting its development; and (b) relieving the disease, i.e., causing regression of the disease or disorder or alleviating symptoms or complications thereof.

The IgG bispecific antibodies of the present invention are expected to treat autoimmune diseases, including IBD (such as CD and UC), Ps0, PsA and HS.

### Pharmaceutical Composition

An IgG bispecific antibody of the present invention can be incorporated into a pharmaceutical composition suitable for administration to a patient. An IgG bispecific antibody of the invention may be administered to a patient alone or with a pharmaceutically acceptable carrier and/or diluent in single or multiple doses. Such pharmaceutical compositions are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions can be designed in accordance with conventional techniques disclosed in, e.g., Remington, The Science and Practice of Pharmacy, 22^{nd} Edition, Loyd V, Ed., Pharmaceutical Press, 2012, which provides a compendium of formulation techniques as are generally known to practitioners. Suitable carriers for pharmaceutical compositions include any material which, when combined with an IgG bispecific antibody of the invention, retains the molecule's activity and is non-reactive with the patient's immune system. A pharmaceutical composition of the present invention comprises an IgG bispecific antibody and one or more pharmaceutically acceptable carriers, diluents or excipients.

A pharmaceutical composition comprising an IgG bispecific antibody of the present invention can be administered to a patient at risk for, or exhibiting, diseases or disorders as described herein using standard administration techniques.

A pharmaceutical composition of the invention contains an “effective” or “therapeutically effective” amount, as used interchangeably herein, of an IgG bispecific antibody of the invention. An effective amount refers to an amount necessary (at dosages and for periods of time and for the means of administration) to achieve the desired therapeutic result. An effective amount of the IgG bispecific antibodies of the present invention may vary according to factors such as the disease state, age, sex, and weight of the individual, and the ability of the antibody or antibody portion to elicit a desired response in the individual. An effective amount is also one in which any toxic or detrimental effect of the IgG bispecific antibody are outweighed by the therapeutically beneficial effects.

### EXAMPLES

### IgG Bispecific Antibody Expression and Purification

An exemplified IgG bispecific antibody of the present invention comprises two HCs and two LCs, wherein the amino acid sequence of HC1 is SEQ ID NO:1, the amino acid sequence of LC1 is SEQ ID NO:2, the amino acid sequence of HC2 is SEQ ID NO:3, and the amino acid sequence of LC2 is SEQ ID NO:4, wherein HC2 comprises threonine at residue 74 of SEQ ID NO:3, and wherein HC1 forms at least one inter-chain disulfide bond with LC1 (forming a HC1-LC1 pair), HC2 forms at least one inter-chain disulfide bond with LC2 (forming a HC2-LC2 pair), and HC1 forms at least two inter-chain disulfide bonds with HC2, and further wherein the HC1-LC1 pair binds to TNFα and the HC2-LC2 pair binds to IL23p19.

The relationship of various regions of the exemplified engineered IgG bispecific antibody of the present invention is presented in Table 1. Additionally, engineered modifications in both the Fab and Fc domains of both the anti-TNFα (HC1-LC1) arm and the anti-IL-23 (HC2-LC2) arm of the exemplified IgG bispecific antibody, which improve chemical and physical stability and drive heterodimeric assembly, are provided in Table 2. Numbering of amino acids residues in Table 1 applies linear numbering; assignment of amino acids to variable domains is based on the International Immunogenetics Information System® available at www.imgt.org; assignment of amino acids to CDR domains applies a combination of the North and Kabat numbering convention and is intended to encompass the broadest amino acid residue coverage afforded by the respective methods. Numbering of amino acids in Table 2 applies linear numbering with corresponding Kabat or EU residue values which may vary from the residue positions of the exemplified IgG bispecific antibody (provided in Table 1) but are applicable for identifying the respective modification positions for example in other IgG bispecific antibodies.

TABLE 1
Amino acid regions of an exemplified engineered
IgG bispecific antibody of the present invention
TNFα arm of the exemplified IgG bispecific antibody

| | |
|---|---|
| HC1 (SEQ ID NO: 1) | LC1 (SEQ ID NO: 2) |

| Region | Positions | Region | Positions |
|---|---|---|---|
| FRH1 | 1-22 | FRL1 | 1 23 |
| HCDR1 | 23-35 | LCDR1 | 24-34 |
| FRH2 | 36-49 | FRL2 | 35-48 |
| HCDR2 | 50-66 | LCDR2 | 49-56 |
| FRH3 | 67-96 | FRL3 | 57-88 |
| HCDR3 | 97-110 | LCDR3 | 89-97 |
| FRH4 | 111-121 | FRL4 | 98-107 |
| CH1 | 122-218 | CL | 108-214 |
| Hinge | 219-234 | ////////////////////////// | //////////////////////// |
| CH2 | 235-344 | ////////////////////////// | //////////////////////// |
| CH3 | 341-450 | ////////////////////////// | //////////////////////// |

IL23p19 arm of the exemplified IgG bispecific antibody

| | |
|---|---|
| HC2 (SEQ ID NO: 3) | LC2 (SEQ ID NO: 4) |

| Region | Positions | Region | Positions |
|---|---|---|---|
| FRH1 | 1-22 | FRL1 | 1-23 |
| HCDR1 | 23-35 | LCDR1 | 24-34 |
| FRH2 | 36-49 | FRL2 | 35-48 |
| HCDR2 | 50-66 | LCDR2 | 49-56 |
| FRH3 | 67-96 | FRL3 | 57-88 |
| HCDR3 | 97-104 | LCDR3 | 89-97 |
| FRH4 | 105-115 | FRL4 | 98-107 |
| CH1 | 116-212 | CL | 108-212 |
| Hinge | 213-228 | ////////////////////////// | ////////////////////////// |
| CH2 | 229-338 | ////////////////////////// | ////////////////////////// |
| CH3 | 339-444 | ////////////////////////// | ////////////////////////// |

TABLE 2
Engineered amino acid modifications in the exemplified IgG
bispecific antibody which improve chemical and physical
stability and drive heterodimeric assembly.
Exemplified IgG Bispecific Antibody

| TNFα arm | | IL23p19 arm | |
|---|---|---|---|
| HC1 | LC1 | HC2 | LC2 |
| (SEQ ID | (SEQ ID | (SEQ ID | (SEQ ID |
| NO. 1) | NO. 2) | NO. 3) | NO. 4) |
| 39Y | 38R | 39K | 1R |
| (39Y Kabat) | (38R Kabat) | (39K Kabat) | (1R Kabat) |
| 113R | 42D | 62E | 38D |
| (105R Kabat) | (42D Kabat) | (62E Kabat) | (38D Kabat) |
| 135C | 122K | 74T | 136Y |
| (127C Kabat) | (122K Kabat) | (74T Kabat) | (135Y Kabat) |
| 222D | ///////////////////// | 166A | 176W |
| 228D Kabat) | | (172A Kabat) | (176W Kabat) |
| 224G | ///////////////////// | 168G | ///////////////////// |
| (230G Kabat) | | (174G Kabat) | |
| 360G | ///////////////////// | 347S | ///////////////////// |
| (356G EU) | | (349S EU) | |
| 361D | ///////////////////// | 364M | ///////////////////// |
| (357D EU) | | (366M EU) | |
| 368Q | ///////////////////// | 368Y | ///////////////////// |
| (364Q EU) | | (370Y EU) | |
| 411A | ///////////////////// | 407V | ///////////////////// |
| (407A EU) | | (409V EU) | |

Furthermore, HC1 and HC2 of the exemplified IgG bispecific antibody are expressed with a lysine at the C-terminal (e.g., residue 451 of HC1, SEQ ID NO:1, and residue 445 of HC2, SEQ ID NO:3). The C-terminal lysine of HC1 and HC2 of the exemplified IgG bispecific antibody may be truncated post-translationally. The C-terminal lysine of HC1 and HC2 may provide a benefit for expression of the IgG bispecific antibodies of the present invention. Additionally, HC2 of the exemplified IgG bispecific antibody is expressed with a glutamine at residue 1 of SEQ ID NO:3. However, the N-terminal glutamine may be converted to pyroglutamic acid during expression.

While engineered modifications disclosed herein are described relative to the germline reference sequences, the skilled person will recognize that alternative germline sequences (e.g., allotype of isotype) may be utilized provided the defined amino acids are located at the indicated positions in the final engineered IgG bispecific antibody according to Kabat or EU residue numbering conventions (as set forth herein).

The exemplified IgG bispecific antibody of the present invention may be expressed and purified essentially as follows. An appropriate host cell, such as HEK 293 or CHO, is either transiently or stably transfected with an expression system for secreting the exemplified IgG bispecific antibody using an optimal predetermined HC:LC vector ratio or a single vector system encoding both HCs (for example, SEQ ID NOs:1 and 3, respectively) and LCs (for example, SEQ ID NOs:2 and 4, respectively). Clarified media, into which the exemplified IgG bispecific antibody has been secreted, is purified using any of many commonly-used techniques. For example, clarified medium, into which the exemplified IgG bispecific antibody has been secreted, is applied to a Protein A affinity column that has been equilibrated with a compatible buffer such as 20 mM TRIS (pH 8.0). The column is washed with 20 mM Tris (pH 7.0) to remove nonspecific binding components followed by a high salt wash to further remove nonspecific components. The column is equilibrated back into 20 mM Tris (pH7.0) and then the bound exemplified IgG bispecific antibody is eluted. for example, by pH step or gradient such as 20 mM citrate (pH 3.0) and neutralized with Tris (pH 8) buffer. The exemplified IgG bispecific antibody is detected by absorbance at 280 nm and collected accordingly. Characterization of the Protein-A captured material results in a titer of 4.94 mg/mL having low LC mis-assembly (3.1%), low HC-LC mispairing composition (3.1%), low half-antibody composition (7.4%), and low HMW polymer (1.5%). Misassembled exemplified IgG bispecific antibody, soluble aggregate, and multimers may be effectively removed by common techniques including hydrophobic interaction chromatography. For instance, the levels of LC mis-assembly and low HMW polymer formation are reduced to 0.6% and 1%, respectively, by use of a second purification column using hydrophobic-interaction chromatography. The exemplified IgG bispecific antibody is concentrated and/or sterile filtered using common techniques. The purity of the exemplified IgG bispecific antibody, after these chromatography steps, may achieve a value greater than 98.0% (monomer). The exemplified IgG bispecific antibody may be immediately frozen at −70° C. or stored at 4° C. for several months.

### Solubility and Stability Analysis of Exemplified IgG Bispecific Antibody

Preliminary studies with the exemplified IgG bispecific antibody of the present invention demonstrate unexpected beneficial properties, including heterodimeric assembly of the IgG bispecific antibody as well as unexpected and beneficial stability, solubility and viscosity properties.

Stability

Stability of the exemplified IgG bispecific antibody is assessed at high concentration (50 and 100 mg/mL) formulated in 10 mM histidine, pH 6.0, ±150 mM NaCl+0.02% (v/v) polysorbate-80. Samples concentrated are incubated for a period of 4 weeks at 25° C. Following incubation, samples are analyzed for (i) percent high molecular weight (% HMW) with size exclusion chromatography (SEC); and (ii) changes in charge profile with capillary isoelectric focusing (cIEF) according to standard procedures. Percent HMW is calculated via Empower analysis of chromatographs and using the ratio of AUC of the peaks eluted before the monomer peak to total AUC. Following analysis, the exemplified IgG bispecific antibody of the present invention resulted in % HMW of less than or equal to 0.6% (0.1% for 50 mg/mL) and change in % of main peak of 1.9%. These results indicate the exemplified IgG bispecific antibody possesses chemical stability similar to, or better than, monovalent antibody therapeutics, and possesses chemical stability sufficient to facilitate development of solution formulations.

Solubility

Sufficiently high solubility is desired to enable required and/or convenient dosing. For example, a 1 mg/kg dose administered by a 1.0 mL injection into a 100 kg patient will require solubility of 100 mg/ml. Solubility of the exemplified IgG bispecific antibody of the present invention is analyzed by concentrating 15 mg of the exemplified IgG bispecific antibody with a 30 KDa molecular weight cut-off filter (for example, Amicon U.C. filters, Millipore, catalog # UFC903024) to a volume of approximately 100 μl. The final concentration of the sample was measured by UV absorbance at A280 using a Cary 50 spectrophotometer (Agilent). Following procedures substantially as described, the exemplified IgG bispecific antibody displays a solubility of greater than 132 mg/ml at pH 6, 10 mM histidine buffer (and greater than 134 mg/mL in PBS, pH 7.4). These results indicate the exemplified IgG bispecific antibody exhibited solubility at or better than monovalent antibody therapeutics, and sufficient to enable high concentration dosing.

Viscosity

Viscosity of the exemplified IgG bispecific antibody is analyzed at 25° C. at an approximate concentration of 100 mg/mL formulated in 10 mM histdine at pH 6.0 including 150 mM NaCl and 0.02% (v/v) polysorbate-80. Viscosity measurements were made in duplicate with a Viscosizer TD capillary instrument (Malvern Instruments). The exemplified IgG bispecific antibody exhibited viscosity, comparable to or better than monovalent antibody therapeutics. of 6.8 cP at approximately 100 mg/mL. By comparison, constructs of the IgG bispecific antibodies of the present invention, wherein threonine at residue 74 of HC2 is engineered to glutamic acid (e.g., T74E, Kabat) as in parental anti-IL-23 antibodies, exhibits viscosity of 17.2 cP (at approximately 100 mg/mL, 25° C.).

### Exemplified IgG Bispecific Antibody Binding Affinity to IL-23 and TNFα

Binding affinity and binding stoichiometry of the exemplified IgG bispecific antibody to human IL-23 and human TNFα is determined using a surface plasmon resonance assay on a Biacore T200 instrument (GE Healthcare, Piscataway N.J.) primed with 1×HBS-EP+(Biacore P/N BR-1006-69) running buffer and analysis temperature set at 37° C. A CM4 chip (Biacore P/N BR-100534) containing immobilized protein A (generated using standard NHS-EDC amine coupling) on all four flow cells is used to employ a capture methodology. IgG bispecific antibody samples are prepared by dilution of 5 μg/mL into running buffer and approximately 100-150 RU is captured on the flow cells. Human IL-23 are prepared at final concentrations of 150.00, 75.00, 37.50, 18.75, 9.38, 4.69, 2.35, 1.18, 0.59 and 0 (blank) nM by dilution into running buffer. Human TNFα are prepared at final concentrations of 50.0, 25.0, 12.5, 6.25, 3.12, 1.56, 0.78, 0.39, 0.19 and 0 (blank) nM by dilution into running buffer.

Each analysis cycle consists of (1) capturing antibody samples on flow cells (Fc3); (2) injection of each human IL-23 concentration over the flow cells at 80 μL/min for 200 seconds followed by return to buffer flow for 900 seconds to monitor dissociation phase; (3) injection of each human TNFα concentration over the flow cells at 80 UL/min for 200 seconds followed by return to buffer flow for 900 seconds to monitor dissociation phase; (4) regeneration of chip surfaces with injection of 10 mM glycine, pH 2.0, for 30 seconds at 50 μL/min over all flow cells; and (5) equilibration of chip surfaces with a 50 μL (60-sec) injection of HBS-EP+ running buffer. Data are processed using standard reference-subtracted method fit to a 1:1 binding model using Biacore T200 Evaluation software, version 1.0, to determine the association rate (kₒₙ, M⁻¹s⁻¹ units), dissociation rate (k_{off}, s⁻¹ units), and RUₘₐₓ (RU units). The equilibrium dissociation constant (K_{D}) is calculated from the relationship K_{D}=k_{off}/kₒₙ, and is in molar units. Results are provided in Table 3.

TABLE 3
Binding affinity to human IL-23 and human TNFα by the
exemplified IgG bispecific antibody at 37° C.

| | kₒₙ | k_{off} | K_{D} | |
|---|---|---|---|---|
| | Avg ± SD | Avg ± SD | Avg ± SD | |
| Antigen | M⁻¹s⁻¹ | s⁻¹ | pM | n |
| Human | 4.12 (±0.15) × 10⁵ | 1.01 ± 0.05 × 10⁻⁴ | 245 ± 8 | 3 |
| IL-23 | | | | |
| Human | 31.0 (±1.2) × 10⁵ | 1.55 ± 0.35 × 10⁻⁴ | 49.6 ± 9.0 | 3 |
| TNFα | | | | |

These results demonstrate that the exemplified IgG bispecific antibody of the present invention binds human IL-23 and human TNFα with high affinity at 37° C.

### Simultaneous Binding of the Exemplified IgG Bispecific Antibody to Human IL-23 and TNFα

A BIAcore T200 instrument is used to determine whether the exemplified IgG bispecific antibody of the present invention can bind human IL-23 and human TNFα simultaneously. Except as noted, all reagents and materials are purchased from GE Healthcare (Piscataway, N.J.). All measurements are performed at 25° C. HBS-EP+ running buffer is used as both the running buffer and sample buffer. Protein A is immobilized on flow cells 1 and 2 of a CM4 sensor chip using an amine coupling kit. The exemplified IgG bispecific antibody is first captured on a flow cell (yielding approximately 95 response units (Δ RU) of exemplified IgG bispecific antibody capture), followed by injection of either human TNFα at 50 nM or human IL-23 at 150 nM for 200 seconds (to saturate binding of the first antigen and an initial RU capture amount was determined). After binding of the first antigen, the other of human TNFα at 50 nM or human IL-23 at 150 nM for 200 seconds is injected (to saturate binding of the second antigen and an additional RU capture amount was determined). One flow cell is maintained as a protein A only control. Chip surface is then regenerated using 10 mM Glycine pH 2. The same process is repeated in a reverse order of the respective antigens. Results demonstrate that the exemplified IgG bispecific antibody of the present invention can bind human IL-23 and human TNFα simultaneously as shown by the increase in response units (initial 18.5 RU from TNFα and then additional 32.5 RU from IL-23) (n=2) from the two ligands binding to the exemplified IgG bispecific antibody.

### Antibody Clearance of Exemplified IgG Bispecific Antibody

Serum pharmacokinetics of the exemplified IgG bispecific antibody are assessed in male Cynomolgus monkeys administered 5 mg/kg of exemplified IgG bispecific antibody (intravenous (N=2); or subcutaneous (N=2)). Exemplified IgG bispecific antibody is prepared in solution of PBS pH 7.2.

Prior to administration, approximately 1.5 mL of blood is collected from each Cynomolgus monkey. Post administration, blood (approximately 1.5 mL) is collected at 1 (intravenous only), 6, 12, 24, 48, 72, 96, 168 and 240 post administration. Blood samples are collected intravenously from a femoral vein into serum separator tubes (e.g., containing no anticoagulant) and processed to serum.

Serum samples are analyzed by total human IgG ELISA utilizing AffiniPure F(ab′)₂ Fragment Goat Anti-Human IgG (Jackson ImmunoResearch Laboratories, Inc.) as capture reagent coated on ELISA plates (Thermo Scientific™ Immulon® 4HBX). Serum samples (100 μL) are added to the individual wells of ELISA plate and incubated at 25° C. for 60 mins. Following incubation, 100 μL (10,000-fold dilution) mouse anti-human IgG Fc-HRP (Southern Biotech) is added to wells of ELISA plate for detection of exemplified IgG bispecific antibody. Unbound enzyme is removed via washing and 100 μL TMB Microwell Peroxidase Substrate System (KPL) is added to individual wells of ELISA plate. Color development is stopped by addition of 100 μL TMB Stop Solution (KPL) and optical density of the wells is measured at 450 nm with wavelength correction set to 630 nm.

A standard curve for the exemplified IgG bispecific antibody is generated by dilution of known amounts of exemplified IgG bispecific antibody into 100% Cynomolgus monkey serum (BioreclamationIVT), followed by 5-fold dilution in blocker casein in PBS (Thermo Scientific™ Pierce™). Standard curve range of exemplified bispecific antibody is 7.8-500 ng/mL.

Pharmacokinetic parameters (clearance values) are calculated using immunoreactivity versus time profile from time zero (administration of exemplified IgG bispecific antibody) to 240 hours post administration (exemplified IgG bispecific antibody) and are determined via non-compartmental analysis using Phoenix (WinNonLin 6.3, Connect 1.3). Following procedures essentially as described, the exemplified IgG bispecific antibody of the present invention possess antibody clearance in Cynomolgus monkey of 0.31 mL/hr/kg (IV) and 0.35 mL/hr/kg (subcutaneous). The results demonstrate that the exemplified IgG bispecific antibody of the present invention has approximately a 5-fold reduced antibody clearance compared to parental IL-23 antibody (described in U.S. Pat. No. 7,872,102) and equivalent antibody clearance compared to an anti-TNF/anti-IL23p19 IgG-scFv bispecific antibody (described in U.S. Patent Publication Number 2016/0122429 A1) in Cynomolgus monkey (results of parental IL-23 antibody and anti-TNF/anti-IL23p19 IgG-scFv bispecific antibody provided in U.S. Patent Publication Number 2016/0122429 A1).

### Effector Function of Exemplified IgG Bispecific Antibody

IgG antibody binding to specific antigens results in opsonization of the target cells which can then recruit and activate either FcγR-bearing immune effector cells or complement proteins via the Fc-portion of IgG. This can result in two distinct Fc-mediated effector function responses: ADCC (antibody-dependent cell-mediated cytotoxicity) and CDC (complement-dependent cytotoxicity), respectively. ADCC involves the release of cytotoxic granules containing lytic enzymes such as perforin and granzymes which cause target cell lysis. CDC involves sequential recruitment and activation of complement proteins that results in formation of membrane attack complex (MAC) leading to target cell lysis. Of the four IgG subclasses expressed by humans, IgG1 and IgG3 are effective in eliciting Fc-mediated effector function response while IgG2 and IgG4 are poor inducers of ADCC and CDC. Exemplified IgG bispecific antibody of the present invention demonstrates ADCC activity at a level comparable to that of parental anti-TNFα antibody (adalimumab, as described in U.S. Pat. No. 6,090,382) and CDC activity at an enhanced level compared to parental anti-TNFα antibody.

ADCC

Surface Plasmon Resonance for FcγR Binding

Binding affinity of the exemplified IgG bispecific antibody to human FcγRI (CD64), FcγRIIa (CD32a), FcγRIIb (CD32b), and FcγRIIIa (CD16a) is compared to the binding affinity of parental anti-TNFα antibody (adalimumab, as described in U.S. Pat. No. 6,090,382) using a surface plasmon resonance assay on a Biacore T200 instrument (GE Healthcare, Piscataway N.J.) primed with 1×HBS-EP+(Biacore P/N BR-1006-69) running buffer and analysis temperature set at 37° C. A CM5 chip (Biacore P/N BR-100-50) containing immobilized protein A (generated using standard NHS-EDC amine coupling) on all four flow cells is used to employ a capture methodology. IgG bispecific antibody samples are prepared by dilution of approximately 5 μg/mL into running buffer and approximately 300 RU is captured on the flow cells. Human FcγRI (produced from transient CHO cells and purified using IMAC and size exclusion chromatography) is prepared at final concentrations of 200.00, 100.00, 50.00, 25.00, 12.50, 6.25, 3.12, 1.56, and 0.78 nM by two-fold serial dilution into running buffer. Human FcγRIIa, FcγRIIb and FcγRIIIa (produced from transient CHO cells and purified using IMAC and size exclusion chromatography) are prepared at final concentrations of 10,000.0, 5,000, 2,500, 1,250, 625, 313, 157, 78, and 39 nM by two-fold serial dilution into running buffer.

Each analysis cycle consists of: (1) capturing antibody samples on flow cells; (2) injection of each human FcγRI, FcγRIIa, FcγRIIb and FcγRIIIa concentration over the flow cells at 40 μL/min for 60 seconds followed by return to buffer flow for 120 seconds to monitor dissociation phase; (3) regeneration of chip surfaces with injection of 10 mM glycine, pH 2.0. for 30 seconds at 50 μL/min over all flow cells; and (4) equilibration of chip surfaces with a 50 μL (60-sec) injection of HBS-EP+ running buffer. Data are processed using standard reference-subtracted method fit to a 1:1 binding model using Biacore T200 Scrubber 2 Evaluation software, version 1.0, to determine the equilibrium dissociation constant (K_{D}) which is calculated from the relationship K_{D}=k_{off}/kₒₙ, and is in molar units. Results are provided in Table 4.

TABLE 4
Binding affinity of exemplified IgG bispecific antibody
and parental anti-TNFα antibody to FcγRI,
FcγRIIa, FcγRIIb and FcγRIIIa.

| | K_{D} Avg ± SD pM (N = 3) | | | |
|---|---|---|---|---|
| | FcγRI | FcγRIIa | FcγRIIb | FcγRIIIa |
| Exemplified | 64.8 ± 2.5 | 0.72 ± 0.05 | 4.00 ± 0.49 | 0.26 ± 0.03 |
| IgG Bispecific | pM | μM | μM | μM |
| Antibody | | | | |
| Adalimumab | 56.3 ± 6.4 | 1.08 ± 0.06 | 5.73 ± 0.21 | 0.43 ± 0.04 |
| | pM | μM | μM | μM |

These results demonstrate that the exemplified IgG bispecific antibody of the present invention binds human FcγRI (CD64), FcγRIIa (CD32a), FcγRIIb (CD32b), and FcγRIIIa (CD16a) at a level comparable to that of parental anti-TNFα antibody.

In Vitro Induction of FcRIIIa-Mediated Effector Function

A CHO cell line stably expressing a non-cleavable form of human membrane TNFα is used to assess the ability of the exemplified IgG bispecific antibody and the parental anti-TNFα antibody (adalimumab, as described in U.S. Pat. No. 6,090,382) to induce Fc-mediated effector function. Briefly, 60 μL of (i) exemplified IgG bispecific antibody (30 μg/mL), or (ii) parental anti-TNFα antibody (30 μg/mL), is diluted with 180 μL assay medium (RPMI 1640 (no phenol red) with 0.1 mM NEAA, 1 mM Sodium Pyruvate, 2 mM L-Glutamine, 100 U/mL Penicillin-Streptomycin, and 0.5% w/v BSA). 50 μL of respective antibody-medium solution is added, in triplicate, to 96 deep-well reaction plates.

CHO cells stably transfecting non-cleavable human TNFα are routinely cultured to a cell density of between 0.2×10⁶ and 3×10⁶ cells/mL. Cells are centrifuged at 400×g for 5 minutes, growth media discarded, and cells are re-suspended in assay media to a final cell density of 1×10⁶ cells/mL. 50 μL of cells is dispensed in each well of the 96 deep-well reaction plate (containing antibody). Plates are incubated at 37° C. for 1 hour.

Jurkat NFAT-FF cell lines stably expressing human FcγRIIa (V158) and NFAT luciferase reporter gene are cultured in RPMI1640 media. Cells are centrifuged at 300×g for 5 minutes, growth media is discarded and cells are re-suspended in assay media to a final cell density of 6×10⁶ cells/mL. 50 μL of cells is added to each well of the 96 deep-well reaction plate (containing previously incubated CHO cells and antibody) and plates are then incubated for another 4 hours at 37° C.

Following incubation, plates are brought to room temperature for 10 minutes followed by addition of 100 μL of One-glo Ex (Promega, E8110) and gentle agitation for one minute. Plates are incubated at room temperature for 10 minutes and luminescence is read using an Enspire Multimode Reader (Perkin Elmer) with per well-read time of 0.1 second. Results are analyzed via Prism v6 (Graph pad). A construct of the exemplified IgG bispecific antibodies of the present invention, wherein threonine at residue 74 of HC2 in the exemplified IgG bispecific antibody is engineered to glutamic acid (e.g., T74E, Kabat, as in parental anti-IL-23 antibodies) is used as an internal standard and assigned an EC50 value of 1. Results (representative of 9 assay runs) are provided in Table 5.

TABLE 5
Induction of FcγRIIIa -mediated Effector Function.

| | | n |
|---|---|---|
| | Relative EC50 | (assay runs) |
| Exemplified IgG | 0.75 ± 0.16 | 9 |
| Bispecific Antibody | | |
| Adalimumab | 0.55 ± 0.12 | 9 |

These results demonstrate that the exemplified IgG bispecific antibody of the present invention demonstrates FcγRIIIa-mediated effector function at a level comparable to that of parental anti-TNFα antibody.

CDC

C1q ELISA Binding Assay

Binding of the exemplified IgG bispecific antibody, as well as parental anti-TNFα antibody (adalimumab as described in U.S. Pat. No. 6,090,382) and IgG1 isotype control antibody, to human complement component C1q is assessed using ELISA. 100 μL of one of (i) exemplified IgG bispecific antibody, (ii) parental anti-TNFα antibody, or (iii) IgG1 isotype control, in DPBS (Dulbecco's HyClone) are added to wells of a 96-well microplate in concentration ranges of 10 μg/mL to 0.19 ug/mL. Plates are incubated overnight at 4° C. with coating agent (to coat wells with antibody) followed with two hours of blocking (casein, 200 μL) at room temperature and then three washes with wash buffer (1×TBE with 0.05% Tween 20).

100 μL of human C1q (10 μg/mL) (MS Biomedical) in casein blocking reagent is added to each well and plates are incubated for 3 hours at room temperature. Following incubation plates are washed three times followed by addition of 100 μL/well (at 1:800 dilution) of Sheep anti-human C1q-HRP (Abcam #ab46191) in casein blocker. Plates are incubated for 1 hour at RT. Following incubation, plates are washed 6 times with wash buffer, and 100 μL/well of TMB Substrate (Pierce) is added to each well. Plates are incubated for 7 minutes followed by addition of 100 μL of 1.0N HC1 to each well to stop the reaction. Optical density is immediately measured using a colorimetric microplate reader set to 450 nm. Following procedures essentially as described, the exemplified IgG bispecific antibody of the present invention, the parental anti-TNFα antibody, and the IgG1 isotype control antibody all exhibit binding to human complement component C1q.

In Vitro Induction of Human Complement-Mediated Effector Function

A CHO cell line stably expressing a non-cleavable form of human membrane TNFα is used to assess the ability of the exemplified IgG bispecific antibody and the parental anti-TNFα antibody (adalimumab as described in U.S. Pat. No. 6,090,382) to induce human complement-mediated effector function. Briefly, 60 μL of (i) exemplified IgG bispecific antibody (30 μg/mL), or (ii) parental anti-TNFα antibody (30 μg/mL), is diluted with 180 μL assay medium (RPMI 1640). 50 μL of respective antibody-medium solution is added, in triplicate, to 96 deep-well reaction plates.

CHO cells stably transfecting non-cleavable human TNFα is cultured to cell density of between 0.2×10⁶ and 3×10⁶ cells/mL. Cells are centrifuged at 300×g for 5 minutes, growth media discarded and cells are re-suspended in assay media to a final cell density of 1×10⁶ cells/mL. 50 uL of cells is dispensed in each well of the 96 deep-well reaction plate (containing antibody). Plates are incubated at 17° C. for 1 hnilr Complement from human serum (Quidel, Cat#A113) is prepared by rapidly thawing at 37° C. and then diluting 1:5 in assay buffer. 50 μL of diluted complement is added to the assay wells and plates are incubated for 2 hours at 37° C. Following incubation, plates are brought to room temperature for 10 minutes followed by addition of 100 μL of Cell Titre Glo (Promega, G775A) and gentle agitation for one minute. Plates are incubated at room temperature for 10 minutes and luminescence is read using an Enspire Multimode Reader (Perkin Elmer) with per well-read time of 0.1 second. Results are analyzed via Prism v6 (Graph pad). Dose response curves are subjected to four parameter logistic curve fit to evaluate EC50 values. A construct of the exemplified IgG bispecific antibodies of the present invention, wherein threonine at residue 74 of HC2 in the exemplified IgG bispecific antibody is engineered to glutamic acid (e.g., T74E, Kabat, as in parental anti-IL-23 antibodies) is used as an internal standard and assigned an EC50 value of 1, wherein the exemplified IgG bispecific antibody demonstrates a relative EC50 value of 0.74±0.05. Parental anti-TNFα antibody did not demonstrate observable CDC activity such that an EC50 value could be calculated. Results are representative of 4 assay runs. Following procedures substantially as described above, anti-TNF/anti-IL23p19 IgG-scFv bispecific antibody (U.S. Patent Publication Number 2016/0122429 A1) also did not demonstrate observable CDC activity at two hours, such that an EC50 value could be calculated.

These results demonstrate that the exemplified IgG bispecific antibody of the present invention, in vitro, demonstrates enhanced human complement-mediated effector function (CDC activity) compared to that of parental anti-TNFα antibody and anti-TNF/anti-IL23p19 IgG-scFv bispecific antibody.

### Immune Complex Formation

Immune complex size (e.g., molecular weight) of antibodies with TNFα has been linked to immunogenic risk. Weight average molecular weight for each of (i.) exemplified IgG bispecific antibody; (ii.) anti-TNF/anti-IL23p19 IgG-scFv bispecific antibody (U.S. Patent Publication Number 2016/0122429 A1); and (iii.) parental anti-TNFα antibody (adalimumab, as described in U.S. Pat. No. 6,090,382), complexed with TNFα, are determined using composition gradient multi-angle light scattering (CG-MALS) instrument (Wyatt Technology, Santa Barbara, Calif.) according to manufacturer instructions. Respective antibody: TNFα complexes are determined at molar ratios of between approximately 0.5 to approximately 5.0, at a fixed antibody concentration of 10 μg/mL, in PBS, at pH 7.2. Following procedures substantially as described herein, the exemplified IgG bispecific antibody, complexed with TNFα, demonstrates a maximum immune complex weight average molecular weight of approximately 400 kDa. Conversely, both anti-TNF/anti-IL23p19 IgG-scFv bispecific antibody and parental anti-TNFα antibody complexed with TNFα, demonstrates a maximum immune complex weight average molecular weight of greater than 1300 kDa. These results demonstrate the exemplified IgG bispecific antibody of the present invention possesses substantially decreased immune complex size in comparison to anti-TNF/anti-IL23p19 IgG-scFv bispecific antibody and parental anti-TNFα antibody.

Immunogenicity Reactivity Assay in Serum from Normal Donors

Pre-existing reactivity of exemplified IgG bispecific antibody, and anti-TNF/anti-IL23p19 IgG-scFv bispecific antibody (U.S. Patent Publication Number 2016/0122429 A1), is assessed in normal human serum (n=60). Immunogenic anti-drug antibody (ADA) reactivity is assessed using the affinity capture elution-bridge format (ACE-Bridge) immunoassay as detailed in Chen et al., Affinity capture elution bridging assay: A novel immunoassay format for detection of anti-therapeutic protein antibodies, J. of Immunological Methods, 431 (2016) 45-51.

Briefly, as described in detail in Chen et al., pre-existing reactivity (i.e. immunoglobulins, complement or other proteins) is assessed in 60 normal human serum samples. Diluted serum is captured on a plate coated with either anti-TNF/anti-IL23p19 IgG-scFv bispecific antibody, or exemplified IgG bispecific antibody, overnight. On the following day, the reactive proteins are eluted with an acid treatment, and neutralized with a master mix that contains Biotin- and ruthenium-labeled anti-TNF/anti-IL23p19 IgG-scFv bispecific antibody or labeled exemplified IgG bispecific antibody. The complexes are then captured again on a streptavidin-coated plate and signal is detected on a Mesoscale platform using ruthenium. Detection in Tier 1 is based on the principle that only bivalent molecules (ADA) will be able to bridge the two labeled reagents and produce a positive signal. The signal is then confirmed in Tier 2, in the presence of an excess of unlabeled anti-TNF/anti-IL23p19 IgG-scFv bispecific antibody added to the detection step. In normal human serum there are no ADA; therefore, pre-existing reactivity should be minimal and fairly close to the background of the assay.

Following procedures substantially as described in Chen et al. and discussed herein, exemplified IgG bispecific antibody demonstrates minimal pre-existing reactivity in normal human serum (Tier 2 cut point of 33.2%), whereas anti-TNF/anti-IL23p19 IgG-scFv bispecific antibody demonstrates significant pre-existing reactivity in normal human serum, with a Tier 2 cut point of 91.9%). These results demonstrate the exemplified IgG bispecific antibody of the present invention possesses decreased pre-existing anti-drug antibody reactivity in normal human serum in vitro than anti-TNF/anti-IL23p19 IgG-scFv bispecific antibody.

Inhibition of IL-23-Mediated Stat 3 Phosphorylation In Vitro from Kit225 Cells

Kit225 cells are a human T cell lymphocytic leukemia cell line that naturally express the IL-23 receptor. The cell line is further engineered to express luciferase under the control of STAT3 binding reporter. Incubation of Kit225 cells with human IL-23 results in the rapid increase of phosphorylation of Stat3 mediated by IL-23R/JAK kinase, which can be measured using commercially available ELISA (e.g., Bright-Glo, Promega, P/N E1501)

Kit225 cells are routinely cultured in assay medium (RPMI 1640 containing 10% FBS, 10 ng/ml human IL-2) (R&D System, P/N 202-IL) and 1× penicillin plus puromycin (200 μg/mL). On the day of assay, the cells are harvested, washed with large volume of serum free RPMI 1640 medium, then resuspended in Opti-MEM I medium at 1×10⁶/mL. 50,000 Kit225 cells per well (in 50 μL) are added to the wells of a U-bottom cultured 96 well plate. The 96 well plate is placed in an incubator and the cells are starved for 3 hours at 37° C., 5% CO₂.

A dose range of the exemplified IgG bispecific antibody from 100 nM to 0.5 pM, with 1:3 dilutions, is evaluated. Each test concentration of exemplified IgG bispecific antibody is pre-incubated with 3 ng/ml recombinant human IL-23 for one hour at 37° C. Assay medium is used for “medium alone” and “medium with 3 ng/ml IL-23” controls. An anti-TNF/anti-IL23p19 IgG-scFv bispecific antibody (U.S. Patent Publication Number 2016/0122429 A1) is used as a positive control in the assay and tested at the same molar range as the bispecific antibody. Control antibodies are also pre-incubated with (3 ng/ml) recombinant human IL-23 for one hour at 37° C. Following pre-incubation, antibody/IL-23 mixtures are transferred to Kit225 cells and incubated for 4 hours at 37° C., 5% CO₂.

At the end of the assay, 1× lysis buffer is added into each well. Final cell lysate is mixed with luciferase assay reagent provided in ELISA (Bright-Glo, Promega P/N E1501). Plates are read according to manufacturer instructions. Results are expressed as the concentration where 50% of the IL-23-induced Stat 3 phosphorylation is inhibited (IC₅₀) by either IgG bispecific antibody or the positive control and is calculated using a 4 parameter sigmoidal fit of the data (GraphPad Prism).

The results demonstrate that the exemplified bispecific antibody of the present invention inhibited human IL-23 induced luciferase activity in the Kit225 cells in a concentration-dependent manner. The inhibition was comparable to that observed with the positive control anti-TNF/anti-IL23p19 IgG-scFv bispecific antibody (with an IC₅₀ for exemplified IgG bispecific antibody of 0.066±0.019 pM, 95% confidence interval, versus 0.097±0.012 pM, 95% confidence interval, for the positive control anti-TNF/anti-IL23p19 IgG-scFv bispecific antibody (average IC₅₀±SEM from three independent experiments)). Negative control antibody did not inhibit Stat 3 phosphorylation in Kit225 cells at any concentration tested. The exemplified IgG bispecific antibody of the present invention effectively neutralized IL-23.

### Inhibition of Soluble TNFα-Induced Cytotoxicity in L929 Cells In Vitro

L929 cells are mouse fibrosarcoma cells that naturally express the TNF receptor. Incubation of L929 cells with human TNFα results in rapid cell death due to excessive formation of reactive oxygen intermediates. Cell death can be measured using MTT cytotoxicity assay, where mitochondrial succinate dehydrogenase in viable cells reduces tetrazolium salt into formazan product, which can be detected with a microplate reader (Molecular Devices SpectriMax 190).

A dose range from 40 nM to 0.002 nM (with three-fold dilution) is evaluated. Each test concentration of exemplified IgG bispecific antibody (100 μL) is added to wells containing 200 μg/mL recombinant human TNFα and 6.25 μg/mL actinomycin-D. Testing is carried out in duplicate wells per treatment. An anti-TNF/anti-IL23p19 IgG-scFv bispecific antibody (U.S. Patent Publication Number 2016/0122429 A1) is used as a positive control in the assay and a human IgG1 isotype control antibody is used as a negative control. Control antibodies are tested at the same molar dose range as exemplified bispecific antibody. Plates containing antibody mixtures are incubated for 60 minutes at room temperature.

L929 cells are routinely cultured in assay medium (1×DMEM Cellgro, 10%/c FBS, 1% Pen-Strep, 1% MEM essential amino acids, 1% L-glutamine, 1% sodium pyruvate). On the day of the assay, the cells are rinsed with 1×PBS (no Ca⁺⁺ or Mg⁺⁺) and detached from culture flasks with 0.25% trypsin+ EDTA. The trypsin is inactivated with assay medium. L929 cells are centrifuged at 215×g for 5 minutes at RT. The cell pellet is resuspended in assay medium. Cell density is measured with a hemocytometer, and 10,000 L929 cells (in 100 μL) are added to 96-well plates and placed in a tissue culture incubator (37° C., 95% relative humidity, 5% CO₂) over night. The antibody/TNFα/actinomycin-D mixture is transferred to the 96 well plates with L929 adherent cells and incubated (37° C., 95% relative humidity, 5% CO₂) 18 hours. The assay medium is removed and the MTT substrate mixture is added to the wells (120 μL). The plates are placed at 37° C., 95% relative humidity, 5% CO₂, for 3 hours. The cell death is determined by reading the plates at 490 nm on a microplate reader (Molecular Devices SpectraMax 190). Results are expressed as the concentration where 50% of the TNFα induced response is inhibited (IC₅₀) (average of three independent experiments±SEM) by either exemplified IgG bispecific antibody or the positive control antibody, calculated using a 4 parameter sigmoidal fit of the data (GraphPad Prism).

Following procedures essentially as described, the exemplified IgG bispecific antibody of the present invention inhibited human TNFα-induced killing of L929 cells in a concentration-dependent manner (IC₅₀=0.32±0.02 nM) comparable to that observed with the positive control (IC₅₀=0.13±0.03 nM). The negative control antibody did not inhibit human TNFα. The results demonstrate the exemplified IgG bispecific antibody of the present invention effectively neutralizes soluble human TNFα.

### Inhibition of Membrane Bound Human TNFα-Induced Cytotoxicity in L929 Cells In Vitro

In order to study the ability of the IgG bispecific antibodies of the present invention to inhibit membrane bound TNFα, known cleavage sites of TNFα are inactivated using a set of mutations previously demonstrated to allow expression of bioactive TNFα on cell surface (Mueller et. al. 1999) in the absence of TNFα cleavage. The non-cleavable TNFα construct is stably transfected to Chinese hamster ovary (CHO) cells. These cells express membrane bound TNFα as shown by flow cytometry. Incubation of L929 cells with CHO cells expressing human non-cleavable bound TNFα results in rapid L929 cell death.

A dose range from 100 nM to 0.005 nM (with three-fold dilution) of antibody is prepared. CHO cells expressing membrane bound human TNFα are routinely maintained in selection medium (AM2001 media, an internal CHO growth media without MSX, 8 mM glutamine, GS supplement, HT supplement with 500 μg/mL G418). On the day of the assay, the cells are counted, rinsed with 1×PBS (no Ca⁺ or Mg⁺⁺), centrifuged at 215×g for 5 min and re-suspended at 50,000 cells/mL in L929 assay medium together with Actinomycin-D (6.25 μg/mL final concentration). 500 cells (in 10 μL) of cell suspension are added to each concentration of antibody mixture. Each test concentration of exemplified IgG bispecific antibody (100 μL) is added to wells containing 500 CHO cells expressing membrane bound human TNFα (in 10 μL) of cell suspension. Testing is carried out in duplicate wells per treatment. An anti-TNF/anti-IL23p19 IgG-scFv bispecific antibody (U.S. Patent Publication Number 2016/0122429 A1) is used as a positive control in the assay and a human IgG1 isotype control antibody is used as a negative control. Control antibodies are tested at the same molar dose range as exemplified bispecific antibody. Plates containing antibody and CHO cell mixture mixtures are incubated for 60 minutes at 37° C., 95% relative humidity, 5% CO₂.

The mixtures containing antibody and membrane bound human TNFα CHO cells are transferred to 96 well plates with L929 adherent cells and incubated 18 hours at 37° C., 95% relative humidity, 5% CO₂. Cell death is measured using an MTT cytotoxicity assay as described above for soluble TNFα L929 assay. Results are expressed as the concentration where 50% of the TNFα induced response is inhibited (IC₅₀) (average of 3 independent experiments±SEM) by either exemplified IgG bispecific or positive control antibody.

Following procedures essentially as described, the exemplified IgG bispecific antibody of the present invention inhibited killing of L929 cells by human non-cleavable membrane bound TNFα CHO cells in a concentration-dependent manner with an IC₅₀ of 3.99±0.57 nM. This inhibition was comparable to that observed with the positive control bispecific antibody (IC₅₀=1.9±0.3 nM), whereas the negative control antibody did not inhibit human TNFα. These results demonstrate the exemplified IgG bispecific antibody of the present invention effectively neutralized membrane bound human TNFα.

Inhibition of Human IL-23-Induced mIL-22 Production In Vivo

Administration of human IL-23 induces expression of mouse IL-22 (mIL-22) in normal Balb C mice in vivo. This human IL-23-induced expression of mIL-22, in vivo, is blocked by the IgG bispecific antibodies of the present invention (which do not cross react with either mouse IL-23 or mouse TNFα).

Normal Balb C mice (N=5 per group), age 7-9 weeks, are injected intraperitoneally with either 50 nmole/kg of exemplified IgG bispecific antibody, anti-TNF/anti-IL23p19 IgG-scFv positive control bispecific antibody (U.S. Patent Publication Number 2016/0122429 A1) or with a negative control antibody (human IgG1 isotype antibody). Three days following injection, the mice are challenged by intraperitoneal injection of 50 nmol/kg of human IL-23. Five hours post IL-23 challenge the mice are sacrificed and plasma is collected. Collected plasma is analyzed by commercial ELISA (eBioscience, Cat.#88-7422-86), according to manufacturer's instructions, for mouse IL-22 expression. Results are provided in Table 6.

TABLE 6
Inhibition of human IL-23-induced mIL-22 production in vivo.

| | Exemplified IgG | | | |
|---|---|---|---|---|
| | Bispecific | Naïve | Negative | Positive |
| | Antibody | mouse | Control | Control |
| mIL22 Level | 13.80 ± 0.66 | 0 ± 0 | 404.0 ± 68.2 | 11.75 ± 0.56 |
| (pg/mL) | | | | |

The results demonstrate that the exemplified IgG bispecific antibody of the present invention blocks the human IL-23-induced increase in mIL-22 expression at a level comparable to that observed with the positive control bispecific antibody. This inhibition is comparable to mouse IL-22 levels observed in naïve mice (p<0.0001, ANOVA followed by Turkey's Multiple Comparison test), whereas the negative control antibody did not inhibit the human IL-23-induced increase in expression of mIL-22. The bispecific antibody of the present invention effectively neutralized human IL-23.

### Inhibition of Human IL-23-Induced Psoriasis In Vivo

Administration of human IL-23 induces psoriasis in normal Balb C mice in vivo. This human IL-23-induced psoriasis, in vivo, is blocked by the IgG bispecific antibodies of the present invention (which do not cross react with either mouse IL-23 or mouse TNFα).

On day zero, day two, and day four, normal Balb C mice (N=5 per group) are injected intradermally with 300 ng of recombinant human IL-23 into the left ear. On day zero, the rhIL23 injected mice were also injected (intraperitoneally) with either (N=5 per group) 100 nmole/kg of exemplified IgG bispecific antibody; anti-TNF/anti-IL23p19 IgG-scFv positive control bispecific antibody (U.S. Patent Publication Number 2016/0122429 A1); or negative control antibody (human IgG1 isotype antibody). On day five the mice are sacrificed and skin sections are hematoxylin and eosin stained. Skin sections are examined microscopically and histopathological findings are recorded as: dermal inflammation; acanthosis; intracorneal inflammation and crusts; and epidermal necrosis and ulceration. Findings are graded 0 (normal); 1 (minimal); 2 (mild); 3 (moderate); or 4 (marked). Total severity score is calculated from the sum of the severity scores for the individual findings. Results are provided in Table 7.

TABLE 7
Inhibition of human IL-23-induced psoriasis in vivo.

| | Exemplified IgG | | | |
|---|---|---|---|---|
| | Bispecific | Naïve | Negative | Positive |
| | Antibody | mouse | Control | Control |
| Severity | 0.60 ± 0.24 | 0.40 ± 0.24 | 4.80 ± 0.49 | 1.00 ± 0.44 |
| Score | | | | |

The results demonstrate that the exemplified IgG bispecific antibody of the present invention blocks human IL-23-induced psoriasis at a level superior to that observed with the positive control bispecific antibody and comparable to naïve mice (p<0.0001, ANOVA followed by Turkey's Multiple Comparison test), whereas the negative control antibody did not inhibit human IL-23-induced psoriasis in the mice.

### Inhibition of Human TNFα-Induced Production of CXCL1 In Vivo

Administration of human TNFα induces a rapid and transient increase of mouse CXCL1 levels in plasma in regular Balb C mice, in vivo. This human TNFα-induced increase of mouse CXCL1 levels, in vivo, is blocked by the IgG bispecific antibodies of the present invention (which do not cross react with either mouse IL-23 or mouse TNFα).

Regular Balb C mice (N=5) are injected intraperitoneally with either: (a) 100 nmole/kg of exemplified IgG bispecific antibody; (b) 100 nmole/kg of anti-TNF/anti-IL23p19 IgG-scFv positive control bispecific antibody (U.S. Patent Publication Number 2016/0122429 A1); or (c) 100 nmole/kg of negative control antibody (human IgG1 isotype antibody). Three days following injection, the mice are challenged by intraperitoneal injection of 18 nmol/kg of human TNFα. Two hours post TNFα challenge the mice are sacrificed and plasma is collected. Collected plasma is analyzed by commercial MSD assay (Masol Scale Discovery, P/N. K152QTG-1), according to manufacturer's instructions, for mouse CXCL1 levels. Results are provided in Table 8.

TABLE 8
Inhibition of human TNFα-induced CXCL1 production in vivo.

| | Exemplified | | | |
|---|---|---|---|---|
| | Bispecific Antibody | Positive Control | Negative Control | Naïve mouse |
| CXCL1 Level | 541.98 ± 132.85 | 682.13 ± 57.39 | 3220.19 ± 340.00 | 49.67 ± 3.30 |
| (pg/mL) | | | | |

The results demonstrate that the exemplified IgG bispecific antibody of the present invention significantly inhibited human TNFα-induced CXCL1 production relative to animals that received the negative control antibody (p<0.0001, ANOVA followed by Turkey's Multiple Comparison test). The reduction in CXCL1 production with the exemplified IgG bispecific antibody was superior to that observed with the positive control. Thus, the exemplified IgG bispecific antibody of the present invention effectively neutralized biological effects induced by human TNFα in mouse.

### Dual Neutralization of Murine IL-23 and Murine TNFα in an Anti-CD40 Antibody-Induced Murine Colitis Model

The IgG bispecific antibodies of the present invention do not bind murine IL-23 (mIL23) or murine TNFα (mTNFα). Therefore, in order to test therapeutic potential of dual targeting TNFα and TL-23 in a rodent model, a surrogate bispecific antibody is generated. The surrogate IgG bispecific is constructed of an engineered chimeric anti-TNFα mIgG2a antibody (engineered chimeric surrogate antibody of Adalimumab targeting equivalent mTNFα epitope, in mouse, as the HC1-LC1 pair of the IgG bispecific antibodies of the present invention targets in human), having fused at the C-terminus of both heavy chains of the anti-TNFα IgG2a antibody an engineered anti-IL-23p19 scFv (engineered from a surrogate murine antibody targeting an equivalent IL23p19 epitope, in mouse, as the HC2-LC2 pair of the IgG bispecific antibody of the present invention targets in human). The binding affinity to mIL23 and mTNFα of the surrogate bispecific is measured, using surface plasmon resonance, to be 1.51 nM and 0.08 nM, respectively.

The therapeutic potential of dual targeting TNFα and IL-23 for colitis is tested in an anti-CD40 antibody-induced murine colitis model by comparing the therapeutic effect (colon weight v. length) of the surrogate bispecific to the therapeutic effect of an anti-TNFα single antibody alone and an anti-IL-23 single antibody alone. Rag1 knockout mice, upon injection of murine anti-CD40 antibody, develop severe, acute colitis which leads to wasting disease, gastrointestinal symptoms including diarrhea and anal inflammation, and weight loss up to 10-20% within 4 days post injection.

To determine the therapeutic potential of dual targeting TNFα and IL-23, three days prior to injection of anti-CD40 antibody, Rag1 knockout mice are administered one of: (a) surrogate bispecific (1.4 mg/kg); (b) anti-TNFα antibody (1 mg/kg); (c) anti-IL-23 antibody (0.3 mg/kg); or (d) control IgG2a antibody (1 mg/kg) (these doses of antibody injection result in similar level of antibody exposure in vivo). Three days following injection, the mice are administered 200 μg per mouse of murine anti-CD40 antibody. Four days after administration of anti-CD40 antibody, the mice are sacrificed and colon weight and length are measured (a ratio of colon weight to length is determined as a measure of colon inflammation greater than mice not administered anti-CD40 antibody). Results are provided in Table 9.

TABLE 9
Change in ratio (colon weight:length).

| | Surrogate | Anti- | Anti- | Murine |
|---|---|---|---|---|
| | bispecific Ab | IL-23 Ab | TNFα Ab | IgG2a Ab |
| Change in colon | 2.68 ± 0.09 | 3.27 ± 0.09 | 3.18 ± 0.09 | 4.0 ± 0.1 |
| weight:length | | | | |
| (mg/mm) | | | | |

The results demonstrate that dual blocking of TNFα and IL-23 by the surrogate bispecific antibody is superior for inhibiting colitis as compared to anti-IL-23 antibody and anti-TNFα antibody therapies alone (p<0.0001 compared to anti-IL-23 antibody alone; p<0.0004 compared to anti-TNF antibody alone, ABOVA followed by comparisons with control using Dunett's method). The inhibition of colitis with the surrogate bispecific was comparable to that observed with the control mice (not administered anti-CD40 antibody). Thus, dual blocking of TNF and IL-23 by the surrogate bispecific effectively inhibits colitis in mouse.

### Dual Neutralization of Murine IL-23 and Murine TNFα in a Murine GPI-Induced Rheumatoid Arthritis Model

DBA/1 mice, upon administration of glucose-6-phosphate-isomerase (GPI), develop rheumatoid arthritis characterized by rapid swelling in the paws. GPI is a protein in the glycolytic pathway and autoantibodies against GPI have been demonstrated in both human rheumatoid arthritis patients as well as murine models. Therapies targeting the TNFα or Th17 pathway alone have demonstrated efficacy in reducing joint swelling in the murine model (Matsumoto 2008, Iwanami 2008).

To demonstrate efficacy of dual neutralization of murine IL-23 and murine TNFα (by surrogate bispecific antibody, described above) when administered in an early therapeutic mode, DBA/1 mice are injected with 400 μg recombinant human GPI and complete Freund's adjuvant (CFA) (1:1 v/v, 2 subcutaneous injection sites at the base of the tail). Eight days following administration of GPI, the mice are administered (by twice weekly intra-peritoneal injection) one of: (a) control murine IgG2a antibody (90 μg); (b) surrogate bispecific (described above) (4.2 μg)+control murine IgG2a antibody (87 μg); (c) surrogate bispecific (12.6 μg)+control murine IgG2a antibody (81 μg); or (d) surrogate bispecific (42 μg)+control murine IgG2a antibody (60 μg). Three weeks (day 21) following administration of GPI, the mice are euthanized.

Starting on the day of administration of GPI (day 0) and days 2, 4, 7, 8, 9, 10, 11, 12, 14, 16, 18, and 21 thereafter, each paw is scored for severity of joint swelling based on a 0-3 scoring system (0=normal; 1=erythema and slight swelling of major joint; 2=moderate to severe swelling of the major joint; and 3=severe swelling of entire paw). The clinical score represents the total score of all 4 paws (a maximum score being 12). The area under curve (AUC) is calculated by trapezoid method for clinical score over time from day 1 to day 21 (the end of study). Clinical score AUC data (shown in Table 10 as mean±SEM) are fitted with a one-way ANOVA model for treatment groups. Test p values of interested comparisons are derived from model based T-test. Results are provided in Table 10.

TABLE 10
Clinical scores for GPI-induced arthritis mice treated
with surrogate bispecific antibody (at various doses) or
control antibody. (p < 0.05 from model based t-test).

| | | Surrogate Bis. | Surrogate Bis. | Surrogate Bis. |
|---|---|---|---|---|
| | | (4.2 μg) + | (12.6 μg) + | (42 μg) + |
| | Control | Control Ab | Control Ab | Control Ab |
| | Ab only | (87 μg) | (81 μg) | (60 μg) |
| Clinical | 105.3 ± 8.4 | 72.8 ± 14.5 | 32.9 ± 10.4 | 33.6 ± 8.9 |
| Score | | | | |
| AUC | | | | |

The results demonstrate that dual blocking of TNFα and IL-23 by surrogate bispecific antibody reduces joint swelling of rheumatoid arthritis in a concentration-dependent manner. On day 9 (the day after treatment initiation with one of the control antibody or surrogate bispecific) mean clinical scores of mice treated with surrogate bispecific Ab are lower than control antibody-treated mice. Maximal clinical scores are attenuated by surrogate bispecific in a dose dependent manner, with clinical score AUCs for all three doses of surrogate bispecific-treated mice significantly lower than control antibody-treated mice (surrogate bispecific concentrations of 12.6 μg and 42 μg achieve the greatest percentage of attenuation).

Comparison of Dual Neutralization of Murine IL-23 and Murine TNFα in a Murine GPI-Induced Rheumatoid Arthritis Model with Neutralization of Murine IL-23 Alone and Murine TNFα Alone

In order to compare efficacy of dual neutralization of murine IL-23 and murine TNFα (by surrogate bispecific antibody) to single murine IL-23 and single murine TNFα treatments when administered in an early therapeutic mode, DBA/1 mice are injected with 400 μg His-tagged recombinant human GPI and CFA (1:1 v/v, 2 subcutaneous injection sites at the base of the tail). Eight days following administration of GPI, the mice are administered (by twice weekly intra-peritoneal injection) one of: (a) control murine IgG2a antibody (30 μg); (b) murine anti-IL-23 antibody (30 μg, engineered from a surrogate murine antibody targeting an equivalent IL23p19 epitope, in mouse, as the HC2-LC2 pair of the IgG bispecific antibody of the present invention targets in human); (c) murine anti-TNFα antibody (30 μg, engineered chimeric surrogate antibody of adalimumab targeting equivalent mTNFα epitope, in mouse, as the HC1-LC1 pair of the IgG bispecific antibodies of the present invention targets in human); or (d) surrogate bispecific (42 μg, described above). Twelve days (day 12) following administration of GPI, the mice are euthanized.

Starting on the day of administration of GPI (day 0) and days 2, 4, 7, 8, 9, 10, 11 and 12 thereafter, each paw is scored for severity of joint swelling based on a 0-3 scoring system (0=normal; 1=erythema and slight swelling of major joint; 2=moderate to severe swelling of the major joint; and 3=severe swelling of entire paw). The clinical score represents the total score of all 4 paws (a maximum score being 12). The area under curve (AUC) is calculated by trapezoid method for clinical score over time from day 8 (immunization with an antibody) to day 12 (end of study). Clinical score AUC data (shown in Table 11 as mean±SEM) are fitted with a one-way ANOVA model for treatment groups. Test p values of interested comparisons are derived from model based T-test. Clinical scores for each individual mouse for days 8 through 12 are fitted with a repeated measurement model with the factors if treatment groups, measurement days and their interaction. Autoregression structure modeling is also applied (to account for correlation of the same animal repeatedly measured across days). Synergy for each is assessed by constructing contrasts on interaction for the Bliss test. Results are provided in Table 11.

TABLE 11
Clinical scores for GPI-induced arthritis mice treated with surrogate
bispecific antibody, murine anti-IL-23 Ab, murine anti-TNFα
Ab, or control antibody. (p < 0.05 from model based t-test).

| | Control | | | Surrogate |
|---|---|---|---|---|
| | mIgG2a Ab | anti-IL-23 Ab | anti-TNFα Ab | bispecific |
| Clinical | 19.7 ± 3.6 | 18.4 ± 2.1 | 19.6 ± 4.1 | 6.1 ± 3.2 |
| Score | | | | |
| AUC | | | | |

The results demonstrate that dual blocking of TNFα and IL-23 by surrogate bispecific antibody is superior for reducing joint swelling of rheumatoid arthritis as compared to anti-IL-23 antibody and anti-TNFα antibody therapies alone. On day 9 (the day after treatment initiation with one of the surrogate bispecific, anti-IL-23 antibody, anti-TNFα antibody or control antibody) mean clinical scores of mice treated with surrogate bispecific are lower than other treatment groups. Thus, dual blocking of TNFα and IL-23, by the surrogate bispecific, effectively reduces joint swelling of rheumatoid arthritis in mouse.

Sequences

Exemplified IgG HC1 (TNFα) (SEQ ID NO: 1)
EVQLVESGGGLVQPGRSLRLSCAASGFTFDDYAMHWVRYAPGKGLEWVSAI
TWNSGHIDYADSVEGRFTISRDNAKNSLYLQMNSLRAEDTAVYYCAKVSYL
STASSLDYWGRGTLVTVSSASTKGPSVFPLAPCSKSTSGGTAALGCLVKDY
FPEPVTVSWNSGALTSGVHTFPAVLQSSGLYSLSSVVTVPSSSLGTQTYIC
NVNHKPSNTKVDKKVEPDSGDKTHTCPPCPAPELLGGPSVFLFPPKPKDTL
MISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPP
SRGDMTKNQVQLTCLVKGFYPSDIAVEWESNGQPENNYKTTPPVLDSDGSF
FLASKLTVDKSRWQQGNVFSCSVMHEALHNHYTQKSLSLSPGX
Wherein Xaa at residue 451 is either K or absent.
Exemplified IgG LC1 (TNFα) (SEQ ID NO: 2)
DIQMTQSPSSLSASVGDRVTITCRASQGIRNYLAWYQRKPGDAPKLLIYAA
STLQSGVPSRFSGSGSGTDFTLTISSLQPEDVATYYCQRYNRAPYTFGQGT
KVEIKRTVAAPSVFIFPPSKEQLKSGTASVVCLLNNFYPREAKVQWKVDNA
LQSGNSQESVTEQDSKDSTYSLSSTLTLSKADYEKBKVYACEVTHQGLSSP
VTKSFNRGEC
Exemplified IgG HC2 (IL23p19) (SEQ ID NO: 3)
XVQLVQSGAEVKKPGSSVKVSCKASGYPFTRYVMHWVRKAPGQGLEWMGYI
NPYNDGVNYNEEFKGRVTITADXSTSTAYMELSSLRSEDTAVYYCARNWDT
GLWGQGTTVTVSSASTKGPSVFPLAPSSKSTSGGTAALGCLVKDYFPEPVT
VSWNSGALTSGVATGPAVLQSSGLYSLSSVVTVPSSSLGTQTYICNVNHKP
SNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTV
LHQDWLNGKEYKCKVSNKALPAPIEKTISKAKGQPREPQVSTLPPSREEMT
KNQVSLMCLVYGFYPSDIAVEWESNGQPENNYKTTPPVLDSDGSFFLYSVL
TVDKSRWQQGNVFSCSVMHEALHNHYTQKSLSLSPGX

wherein Xaa at residue 1 is either Q or pyroglutamic acid; Xaa at residue 74 is either T or E; and Xaa at residue 445 is either K or absent.

Exemplified IgG LC2 (IL23p19)
(SEQ ID NO: 4)
RIQMTQSPSSLSASVGDRVTITCKASDHIGKELTWYQDKPGKAPKWYGATSKL
TGVPSRFSGSGSGTDFTLTISSLQPEDFATYYCQQYWSTPFTFGGGTKVEIKGQPK
AAPSVTLFPPSSEELQANKATLVCYISDFYPGAVTVAWKADSSPVKAGVETTTPS
KQSNNKYAAWSYLSLTPEQWKSHRSYSCQVTHEGSTVEKTVAPTEC
DNA Seq. Encoding Exemplified IgG HC1
(SEQ ID NO: 5)
atggagacggacactctgctcctgtgggtgctcctgctttgggtaccgggttcaacgggagaggtgcagctggtggagtctggg
ggaggcttggtacagcctgggaggtccctgagactctcctgtgcagcctaggattcaccatgatgactatgccatgcactgggt
ccgctacgctccagggaaggggctggagtgggtgtcagctattacttggaatagtggtcacatagactacgcagactccgtgga
gggccggttcaccatctccagagacaatgccaagaactccctgtatctgcaaatgaacagcctgagagccgaggacacggcc
gtatattactgtgcgaaagtgagctacctgagtactgcctccagcctggactactggggcagaggaaccctggtgaccgtcagct
cagctagcaccaagggcccatcggtcttccccctggcaccctgctccaagagcacctctgggggcacagcggccctgggctg
cctggtcaaggactacttccccgaaccggtgacggtgtcgtggaactcaggcgccctgaccagcggcgtgcacaccttcccgg
ctgtcctacagtcctcaggactctactccctcagcagcgtggtgaccgtgccctccagcagcttgggcacccagacctacatctg
caacgtgaatcacaagcccagcaacaccaaggtggacaagaaagttgagcccgactctggcgacaaaactcacacatgccca
ccgtgcccagcacctgaactcctggggggaccgtcagtcttcctcttccccccaaaacccaaggacaccctcatgatctcccgg
acccctgaggtcacatgcgtggtggtggacgtgagccacgaagaccctgaggtcaagttcaactggtacgtggazggcgtgg
aggtgcataatgccaagacaaagccgcgggaggagcagtacaacagcacgtaccgtgtggtcagcgtcctcaccgtcctgca
ccaggactggctgaatggcaaggagtacaagtgcaaggtctccaacaaagccctcccagcccccatcgagaaaaccatctcc
aaagccaaagggcagccccgagaaccacaggtgtacaccctgcccccatcccggggggacatgaccaagaaccaggtcca
gctgacctgcctggtcaaaggcttctatcccagcgacatcgccgtggagtgggagagcaatgggcagccggagaacaactac
aagaccacgcctcccgtgctggactccgacggctccttcttcctcgccagcaagctcaccgtggacaagagcaggtggcagca
ggggaacgtcttctcatgctccgtgatgcatgaggctctgcacaaccactacacgcagaagagcctctccctgtctccgggtaaa
DNA Seq. Encoding Exemplified IgG LC1
(SEQ ID NO: 6)
atggaaactgacaccctgctgctctgggtactgctcctttgggacctgggagcacaggcgacatccagatgacccagtctccat
cctccctgtctgcatctgtaggagacagagtcaccatcacttgccgggcgagtcagggcattcgcaattatttagcctggtatcag
cggaaaccaggggacgctcctaagctcctgatctatgctgcatccactttgcaatcaggggtcccatctcggttcagtggcagtg
gatctgggacagatttcactctcaccatcagcagcctgcagcctgaagatgttgcaacttattactgtcaacgctataaccgtgccc
cttacacgttcggccaagggaccaaggtggagatcaagcgtacggtggctgcaccatctgtcttcatcttcccgccatctaagga
gcagttgaaatctggaactgcctctgttgtgtgcctgctgaataacttctatcccagagaggccaaagtacagtggaaggtggata
acgccctccaatcgggtaactcccaggagagtgtcacagagcaggacagcaaggacagcacctacagcctcagcagcaccc
tgacgctgagcaaagcagactacgagaaacacaaagtctacgcctgcgaagtcacccatcagggcctgagctcgcccgtcac
aaagagcttcaacaggggagagtgc
DNA Seq. Encoding Exemplified IgG HC2
(SEQ ID NO: 7)
atggaaaccgatacgctcctgctgtgggttctcctcttgtgggtccccggctctaccgggcaggtgcagctggtgcagtctgggg
ctgaggtgaagaagcctgggtcctcggtgaaggtctcctgcaaggcttctggatatcccttcactcgttatgttatgcactgggtgc
gaaaggcccctggacaagggcttgagtggatgggatatattaatccttacaatgatggtgtgaactacaatgaggagttcaaagg
cagagtcacgattaccgcggacacctccacgagcacagcctacatggagctgagcagcctgagatctgaggacacggccgtg
tattactgtgcgagaaactgggacacaggcactggggccaaggcaccactgtcacagtctcctcagctagcaccaagggccc
atcggtcttccccctggcaccctcctccaagagcacctctgggggcacagcggccctgggctgcctggtcaaggactacttccc
cgaaccggtgacggtgtcgtggaactcaggcgccctgaccagcggcgtggccaccggcccggctgtcctacagtcctcagga
ctctactccctcagcagcgtggtgaccgtgccctccagcagcttgggcacccagacctacatctgcaacgtgaatcacaagccc
agcaacaccaaggtggacaagaaagttgagcccaaatcttgtgacaaaactcacacatgcccaccgtgcccagcacctgaact
cctggggggaccgtcagtcttcctcttccccccaaaacccaaggacaccctcatgatctcccggacccctgaggtcacatgcgt
ggtggtggacgtgagccacgaagaccctgaggtcaagttcaactggtacgtggacggcgtggaggtgcataatgccaagaca
aagccgcgggaggagcagtacaacagcacgtaccgtgtggtcagcgtcctcaccgtcctgcaccaggactggctgaatggca
aggagtacaagtgcaaggtctccaacaaagccctcccagcccccatcgagaaaaccatctccaaagccaaagggcagcccc
gagaaccacaggtgagcaccctgcccccatcccgggaggagatgaccaagaaccaggtcagcctgatgtgcctggtctacgg
cttctatcccagcgacatcgccgtggagtgggagagcaatgggcagccggagaacaactacaagaccacgcctcccgtgctg
gactccgacggctccttcttcctctacagcgtgctcaccgtggacaagagcaggtggcagcaggggaacgtcttctcatgctcc
gtgatgcatgaggctctgcacaaccactacacgcagaagagcctctccctgtctccgggtaaa
DNA Seq. Encoding Exemplified IgG LC2
(SEQ ID NO: 8)
atggagacagacacactcctgctatgggtactgctgctctgggttccaggatccactggtcgcatccagatgacccagtctccat
cctccctgtctgcatctgtaggagacagagtcaccatcacttgcaaggcaagtgaccacattggcaaatttttaacttggtatcagg
acaaaccagggaaagcccctaagctcctgatctatggtgcaaccagtaagttgactggggtcccatcaaggttcagtggcagtg
gatctgggacagatttcactctcaccatcagcagtctgcaacctgaagattttgcaacttactactgtcaacagtattggagtactcc
gttcacgttcggaggggggaccaaggtggaaataaaaggtcagcccaaggctgccccctcggtcactctgttcccgccctcctc
tgaggagatcaagccaacaaggccacactggtgtgttacataagtgacttctacccgggagccgtgacagtggcctggaagg
cagatagcagccccgtcaaggcgggagtggagaccaccacaccctccaaacaaagcaacaacaagtacgcggcctggagct
atctgagcctgacgcctgagcagtggaagtcccacagaagctacagctgccaggtcacgcatgaagggagcaccgtggaga
agacagtggcccctacagaatgc
Exemplified IgG HCVR1
(SEQ ID NO: 9)
EVQLVESGGGLVQPGRSLRLSCAASGFTFDDYAMHWVRYAPGKGLEWVSAITW
NSGHIDYADSVEGRFTISRDNAKNSLYLQMNSLRAEDTAVYYCAKVSYLSTASS
LDYWGRGTLVTVSS
Exemplified IgG LCVR1
(SEQ ID NO: 10)
DIQMTQSPSSLSASVGDRVTITCRASQGLRNYLAWYQRKPGDAPKLLIYAASTLQ
SGYPSRFSGSGSGTDFTLTISSLQPEDVATYYCQRYNRAPYTFGQGTKVELK
Exemplified IgG HCVR2
(SEQ ID NO: 11)
XVQLVQSGAEVKKPGSSVKVSCKASGYPFTRYVMHWVRKAPGQGLEWMGYIN
PYNDGVNYNEEFKGRVTITADXSTSTAYMELSSLRSEDTAVYYCARNWDTGLW
GQGTTVTVSS
wherein Xaa at residue 1 is either Q or pyroglutamic acid, and Xaa at residue
74 is either T or E.
Exemplified IgG LCVR2
(SEQ ID NO: 12)
RIQMTQSPSSLSASVGDRVTITCKASDHIGKFLTWYQDKPGKAPKLLIYGATSKL
TGVPSRFSGSGSGTDFTLTISSLQPEDFATYYCQQYWSTPFTFGGGTKVEIK
Exemplified HCDR1
(SEQ ID NO: 13)
AASGFTFDDYAMH
Exemplified HCDR2
(SEQ ID NO: 14)
AITWNSGHIDYADSVEG
Exemplified HCDR3
(SEQ ID NO: 15)
AKVSYLSTASSLDY
Exemplified LCDR1
(SEQ ID NO: 16)
RASQGIRNYLA
Exemplified LCDR2
(SEQ ID NO: 17)
YAASTLQS
Exemplified LCDR3
(SEQ ID NO: 18)
QRYNRAPYT
Exemplified HCDR4
(SEQ ID NO: 19)
KASGYPFTRYVMH
Exemplified HCDR5
(SEQ ID NO: 20)
YINPYNDGVNYNEEFKG
Exemplified HCDR6
(SEQ ID NO: 21)
ARNWDTGL
Exemplified LCDR4
(SEQ ID NO: 22)
KASDHIGKFLT
Exemplified LCDR5
(SEQ ID NO: 23)
YGATSKLT
Exemplified LCDR6
(SEQ ID NO: 24)
QQYWSTPFT

We claim:

1. An immunoglobulin G (IgG) bispecific antibody comprising,
a.) a first heavy chain (HC1) comprising a heavy chain variable region (HCVR1), wherein HCVR1 comprises heavy chain complementarity determining regions (HCDR) 1, 2 and 3, wherein the amino acid sequence of HCDR1 is SEQ ID NO:13, the amino acid sequence of HCDR2 is SEQ ID NO:14 and the amino acid sequence of HCDR3 is SEQ ID NO:15;
b.) a first light chain (LC1) comprising a light chain variable region (LCVR1), wherein LCVR1 comprises light chain complementarity determining regions (LCDR) 1, 2 and 3, wherein the amino acid sequence of LCDR1 is SEQ ID NO:16, the amino acid sequence of LCDR2 is SEQ ID NO:17 and the amino acid sequence of LCDR3 is SEQ ID NO: 18;
c.) a second heavy chain (HC2) comprising a heavy chain variable region (HCVR2), wherein HCVR2 comprises HCDRs 4, 5 and 6, wherein the amino acid sequence of HCDR4 is SEQ ID NO:19, the amino acid sequence of HCDR5 is SEQ ID NO:20 and the amino acid sequence of HCDR6 is SEQ ID NO: 21, and
d.) a second light chain (LC2) comprising a light chain variable region (LCVR2), wherein LCVR2 comprises LCDRs 4, 5 and 6, wherein the amino acid sequence of LCDR4 is SEQ ID NO:22, the amino acid sequence of LCDR5 is SEQ ID NO:23 and the amino acid sequence of LCDR6 is SEQ ID NO: 24,
wherein HC1 forms at least one inter-chain disulfide bond with LC1, HC2 forms at least one inter-chain disulfide bond with LC2, and HC1 forms at least two inter-chain disulfide bond with HC2, and wherein the IgG bispecific antibody binds to human TNF alpha (TNFα) and the p19 subunit of human IL-23 (IL23p19).

2. The IgG bispecific antibody of claim 1, wherein HC2 comprises threonine at residue 74 (Kabat).

3. The IgG bispecific antibody of claim 1, wherein
a) HC1 comprises tyrosine at residue 39 (Kabat), arginine at residue 105 (Kabat), cysteine at residue 127 (Kabat), aspartic acid at residue 228 (Kabat), and glycine at residue 230 (Kabat),
b) LC1 comprises arginine at residue 38 (Kabat), aspartic acid at residue 42 (Kabat), and lysine at residue 122 (Kabat),
c) HC2 comprises lysine at residue 39 (Kabat), glutamic acid at residue 62 (Kabat), alanine at residue 172 (Kabat), and glycine at residue 174 (Kabat), and
d) LC2 comprises arginine at residue 1 (Kabat), aspartic acid at residue 38 (Kabat), tyrosine at residue 135 (Kabat), and tryptophan at residue 176 (Kabat).

4. The IgG bispecific antibody of claim 3, wherein the amino acid sequence of HCVR1 is SEQ ID NO:9, the amino acid sequence of LCVR1 is SEQ ID NO:10, the amino acid sequence of HCVR2 is SEQ ID NO:11, and the amino acid sequence of LCVR2 is SEQ ID NO:12.

5. The IgG bispecific antibody of claim 4, wherein HCVR2 comprises a threonine at residue 74 of SEQ ID NO:11.

6. The IgG bispecific antibody of claim 1, wherein HC1 and HC2 are human IgG1 heavy chains, LC1 is a human kappa light chain, and LC2 is a human kappa light chain variable domain and a human lambda light chain constant region.

7. The IgG bispecific antibody of claim 6, wherein
a) HC1 comprises glycine at residue 356 (EU), aspartic acid at residue 357 (EU), glutamine at residue 364 (EU), and alanine at residue 407 (EU), and
b) HC2 comprises serine at residue 349 (EU), methionine at residue 366 (EU), tyrosine at residue 370 (EU), and valine at residue 409 (EU).

8. The IgG bispecific antibody of claim 7, wherein the amino acid sequence of HC1 is SEQ ID NO:1, the amino acid sequence of LC1 is SEQ ID NO:2, the amino acid sequence of HC2 is SEQ ID NO:3 and the amino acid sequence of LC2 is SEQ ID NO:4.

9. The IgG bispecific antibody of claim 8, wherein HC2 comprises a threonine at residue 74 of SEQ ID NO:3.

10. A method of treating an autoimmune disease comprising administering to a patient in need thereof a therapeutically effective amount of an IgG bispecific antibody comprising,
a.) a first heavy chain (HC1) comprising a heavy chain variable region (HCVR1), wherein HCVR1 comprises heavy chain complementarity determining regions (HCDR) 1, 2 and 3, wherein the amino acid sequence of HCDR1 is SEQ ID NO:13, the amino acid sequence of HCDR2 is SEQ ID NO:14 and the amino acid sequence of HCDR3 is SEQ ID NO:15;
b.) a first light chain (LC1) comprising a light chain variable region (LCVR1), wherein LCVR1 comprises light chain complementarity determining regions (LCDR) 1, 2 and 3, wherein the amino acid sequence of LCDR1 is SEQ ID NO:16, the amino acid sequence of LCDR2 is SEQ ID NO:17 and the amino acid sequence of LCDR3 is SEQ ID NO: 18;
c.) a second heavy chain (HC2) comprising a heavy chain variable region (HCVR2), wherein HCVR2 comprises HCDRs 4, 5 and 6, wherein the amino acid sequence of HCDR4 is SEQ ID NO:19, the amino acid sequence of HCDR5 is SEQ ID NO:20 and the amino acid sequence of HCDR6 is SEQ ID NO: 21, and
d.) a second light chain (LC2) comprising a light chain variable region (LCVR2), wherein LCVR2 comprises LCDRs 4, 5 and 6, wherein the amino acid sequence of LCDR4 is SEQ ID NO:22, the amino acid sequence of LCDR5 is SEQ ID NO:23 and the amino acid sequence of LCDR6 is SEQ ID NO: 24,
wherein HC1 forms at least one inter-chain disulfide bond with LC1, HC2 forms at least one inter-chain disulfide bond with LC2, and HC1 forms at least two inter-chain disulfide bond with HC2, and wherein the IgG bispecific antibody binds to human TNF alpha (TNFα) and the p19 subunit of human IL-23 (IL23p19).

11. The method of claim 10, wherein:
e) HC1 further comprises tyrosine at residue 39 (Kabat), arginine at residue 105 (Kabat), cysteine at residue 127 (Kabat), aspartic acid at residue 228 (Kabat), and glycine at residue 230 (Kabat),
f) LC1 further comprises arginine at residue 38 (Kabat), aspartic acid at residue 42 (Kabat), and lysine at residue 122 (Kabat),
g) HC2 further comprises lysine at residue 39 (Kabat), glutamic acid at residue 62 (Kabat), alanine at residue 172 (Kabat), and glycine at residue 174 (Kabat), and
h) LC2 further comprises arginine at residue 1 (Kabat), aspartic acid at residue 38 (Kabat), tyrosine at residue 135 (Kabat), and tryptophan at residue 176 (Kabat).

12. The method of claim 11, wherein the amino acid sequence of HCVR1 is SEQ ID NO:9, the amino acid sequence of LCVR1 is SEQ ID NO:10, the amino acid sequence of HCVR2 is SEQ ID NO:11, and the amino acid sequence of LCVR2 is SEQ ID NO:12.

13. The method of claim 12, wherein HC1 and HC2 are human IgG1 heavy chains, LC1 is a human kappa light chain, and LC2 is a human kappa light chain variable domain and a human lambda light chain constant region, and further wherein HCVR2 comprises a threonine at residue 74 of SEQ ID NO:11.

14. The method of claim 13, wherein the amino acid sequence of HC1 is SEQ ID NO:1, the amino acid sequence of LC1 is SEQ ID NO:2, the amino acid sequence of HC2 is SEQ ID NO:3 and the amino acid sequence of LC2 is SEQ ID NO:4.

15. The method of claim 10, wherein the autoimmune disease is inflammatory bowel disease.

16. The method of claim 15, wherein the inflammatory bowel disease is one of Crohn's disease and ulcerative colitis.

17. The method of claim 10, wherein the autoimmune disease is one of psoriasis, psoriatic arthritis, and Hidradenitis suppurativa.

18. The method of claim 14, wherein the autoimmune disease is one of inflammatory bowel disease, psoriasis, psoriatic arthritis, and Hidradenitis suppurativa.

19. A pharmaceutical composition comprising an IgG bispecific antibody and one or more pharmaceutically acceptable carriers, diluents, or excipients, wherein the IgG bispecific antibody comprises,
a.) a first heavy chain (HC1) comprising a heavy chain variable region (HCVR1), wherein HCVR1 comprises heavy chain complementarity determining regions (HCDR) 1, 2 and 3, wherein the amino acid sequence of HCDR1 is SEQ ID NO:13, the amino acid sequence of HCDR2 is SEQ ID NO:14 and the amino acid sequence of HCDR3 is SEQ ID NO:15;
b.) a first light chain (LC1) comprising a light chain variable region (LCVR1), wherein LCVR1 comprises light chain complementarity determining regions (LCDR) 1, 2 and 3, wherein the amino acid sequence of LCDR1 is SEQ ID NO:16, the amino acid sequence of LCDR2 is SEQ ID NO:17 and the amino acid sequence of LCDR3 is SEQ ID NO: 18;
c.) a second heavy chain (HC2) comprising a heavy chain variable region (HCVR2), wherein HCVR2 comprises HCDRs 4, 5 and 6, wherein the amino acid sequence of HCDR4 is SEQ ID NO:19, the amino acid sequence of HCDR5 is SEQ ID NO:20 and the amino acid sequence of HCDR6 is SEQ ID NO: 21, and
d.) a second light chain (LC2) comprising a light chain variable region (LCVR2), wherein LCVR2 comprises LCDRs 4, 5 and 6, wherein the amino acid sequence of LCDR4 is SEQ ID NO:22, the amino acid sequence of LCDR5 is SEQ ID NO:23 and the amino acid sequence of LCDR6 is SEQ ID NO: 24,
wherein HC1 forms at least one inter-chain disulfide bond with LC1, HC2 forms at least one inter-chain disulfide bond with LC2, and HC1 forms at least two inter-chain disulfide bond with HC2, and wherein the IgG bispecific antibody binds to human TNF alpha (TNFα) and the p19 subunit of human IL-23 (IL23p19).

20. The pharmaceutical composition of claim 19, wherein the amino acid sequence of HC1 is SEQ ID NO:1, the amino acid sequence of LC1 is SEQ ID NO:2, the amino acid sequence of HC2 is SEQ ID NO:3 and the amino acid sequence of LC2 is SEQ ID NO:4.
